(19) United States
(12) Patent Application Publication
(10) Pub. No.: US 2008/0254058 A1
(43) Pub. Date: Oct. 16, 2008

(54) Pharmaceutical Composition Comprising a Bacterial Cell Displaying a Heterologous Proteinaceous Compound

(75) Inventors: Jacob Glenting, Copenhagen (DK); Flemming Jorgensen, Lyngby (DK); Soren Michael Madsen, Copenhagen K (DK); Hans Israelsen, Allerod (DK)

Correspondence Address: DARBY & DARBY P.C., P.O. BOX 770, Church Street Station, New York, NY, 10008-0770, US

(73) Assignee: ALK-ABELLO A/S, Horsholm (DK)

(21) Appl. No.: 11/721,246

(22) PCT Filed: Dec. 14, 2005
(86) PCT No.: PCT/DK05/00792

§ 371 Date: Aug. 6, 2007

Related U.S. Application Data
(60) Provisional application No. 60/635,762, filed on Dec. 14, 2004.

(30) Foreign Application Priority Data
Dec. 14, 2004 (DK) PA200401935

(51) Int. Cl. A61K 39/385; A61P 37/08; A61P 35/00; A61P 31/00

(52) U.S. Cl. 424/197.11; 424/93.45; 424/93.44; 424/275.1

(57) ABSTRACT

The present invention pertains to a composition for the manufacture of a medicament comprising living or dead bacteria with controlled amounts of surface-coupled proteins or proteinaceous compounds and a method for the preparation of the composition. The bacterium provides a multivalent heterologous protein display vehicle that may be used in the manufacture of vaccines or medicaments for delivery via the mucosa.

### BACKGROUND OF THE INVENTION

In recent years, mucosal vaccination has received increasing attention due to i) new insights into the mechanisms of the immune system, ii) the rationale of mimicking the route of infection for a majority of pathogens, but also due to iii) the need for easily administered and effective vaccines against new and emerging diseases. Furthermore, the global threat of bio-terror calls for effective vaccines, which can be produced easily and administered quickly without trained personnel.

The mucosal immune system appears to be ideal for obtaining effective immune responses, since induction at one site of the mucosa results in a specific response throughout the mucosal immune system. Induction at mucosal sites most often also results in a systemic immune response (Huang J. et al., 2004 Vaccine 6:794-801; Verdonck F. et al., 2004 Vaccine 31-32:4291-9). Most pathogens infect their hosts via the mucosal surfaces. This fact makes it advantageous to create vaccines that exert their effect at an early stage of this infection route. Consequently, effort has been focused on the development of non-pathogenic or attenuated pathogenic microorganisms that are able to deliver specific vaccine components towards pathogens.

Significant progress, during recent decades, has been made in developing methods for surface display of heterologous proteins using recombinant microorganisms. Surface display of heterologous proteins has been shown in both attenuated pathogenic bacteria such as Salmonella (Arnold H. et al. 2004 Infect Immun. 11:6546-53) and non-pathogen bacteria such as Staphylococcus (Wernerus H. et al. 2002 Biotechnol J. 1:67-78) or Lactobacillus using recombinant DNA technology (Grangette C. et al., 2004 Infect Immun. 5:2731-7). The heterologous proteins are produced by the recombinant cell, which in turn direct the protein to the cell surface. Several methods have been described to surface-anchor the secreted protein in microorganisms. One approach, shown in Staphylococcus, is to introduce a stretch of cell wall anchoring amino acids into the secreted protein. The chimeric protein is integrated into the cell wall during its secretion, where it binds to the cell wall through the stretch of anchoring amino acids (Wernerus H. et al. 2002 Biotechnol J. 1:67-78). In an alternative approach bacterial ghosts produced by cell lysis, have been employed as a carrier or targeting vehicle for active substances such as antibodies or therapeutically effective polypeptides (CA 2,370,714). Suitable bacterial strains comprising a lytic gene e.g. bacteriophage gene E, are induced to undergo cell lysis to form an empty ghost. The desired active substance is then transported into the empty ghost, where it may be immobilised at the inner cell membrane surface. In this case the active substance is encapsulated in the interior of the cell ghost, rather than exposed on the cell surface.

Several publications describe the significant biological potential of using the surface display properties of live bacteria for vaccine delivery technology (Wernerus H. et al. 2004 Biotechnol Appl Biochem. 40 (3): 209-28). However these approaches rely on recombinant microorganisms, where the risk of, and general opposition to using and releasing genetically modified organisms has been a barrier to applying this live vaccine delivery technology in humans and animals. Killed bacteria that contain recombinant DNA are also considered a risk, since they carry recombinant DNA that eventually may be spread in the environment. Under certain circumstances, as for example under a bio-terrorist attack, the use of GMO-based technology may be regarded as an acceptable risk.

Very few attempts have been made to provide an antigen-display vehicle that is not dependent on genetically modified bacterial strains. US patent application US 2003/0180816 A1 discloses a method for obtaining cell-wall material from non-GM Gram-positive bacteria with improved capacity for binding proteins that are fused to an AcmA cell-wall binding domain (WO99/25836). According to the disclosed method, Gram-positive bacteria are treated with an acidic solution to remove cell-wall components including proteins, lipoteichoic acid and carbohydrates. The resulting cell-wall material is thus largely stripped of native proteins, but remains a barrier against the exterior environment, and is designated a ghost. Chimeric proteins, comprising the AcmA domain protein, can be bound in a non-covalent manner to these ghosts.

Improved modes of antigen and allergen presentation are required in order to meet the needs of vaccination procedures designed to provide treatment for both patients suffering from allergy as well as those suffering from an infectious disease. The concept of vaccination is based on two fundamental characteristics of the immune system, namely specificity and memory. Vaccination will prime the immune system of the recipient, and upon repeated exposure to similar proteins the immune system will be in a position to respond more rigorously to the challenge of for example a microbial infection. Vaccines are mixtures of proteins intended to be used in vaccination for the purpose of generating such a protective immune response in the recipient. The protection will comprise only components present in the vaccine and homologous antigens.

Compared to other types of vaccination, allergy vaccination is complicated by the existence of an ongoing immune response in allergic patients. This immune response is characterised by the presence of allergen specific IgE mediating the release of allergic symptoms upon exposure to allergens. Thus, allergy vaccination using allergens from natural sources has an inherent risk of side effects being in the utmost consequence life threatening to the patient.

Approaches to circumvent this problem may be divided in three categories. In practise measures from more than one category are often combined. First category of measures includes the administration of several small doses over prolonged time to reach a substantial accumulated dose. Second category of measures includes physical modification of the allergens by incorporation of the allergens into gel substances such as aluminium hydroxide. Aluminium hydroxide formulation has an adjuvant effect and a depot effect of slow allergen release reducing the tissue concentration of active allergen components. Third category of measures include chemical modification of the allergens for the purpose of reducing allergenicity, i.e. IgE binding.

Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and in addition, most patients experience a relief in disease severity and symptoms experienced, or at least an arrest in disease aggravation. Thus, specific allergy vaccination has preventive effects reducing the risk of hay fever developing into asthma, and reducing the risk of developing new sensitivities.

The immunological mechanism underlying successful allergy vaccination is not known in detail. A specific immune response, such as the production of antibodies against a particular pathogen, is known as an adaptive immune response. This response can be distinguished from the innate immune response, which is an unspecific reaction towards pathogens. An allergy vaccine is bound to address the adaptive immune response, which includes cells and molecules with antigen specificity, such as T-cells and the antibody producing B-cells. B-cells cannot mature into antibody producing cells without help from T-cells of the corresponding specificity. T-cells that participate in the stimulation of allergic immune responses are primarily of the Th2 type. Establishment of a new balance between Th1 and Th2 cells has been proposed to be beneficial and central to the immunological mechanism of specific allergy vaccination. Whether this is brought about by a reduction in Th2 cells, a shift from Th2 to Th1 cells, or an up-regulation of Th1 cells is controversial. Recently, regulatory T-cells have been proposed to be important for the mechanism of allergy vaccination. According to this model regulatory T-cells, i.e. Th3 or Tr1 cells, down-regulate both Th1 and Th2 cells of the corresponding antigen specificity. In spite of these ambiguities it is generally believed that an active vaccine must have the capacity to stimulate allergen specific T-cells, preferably TH1 cells.

Specific allergy vaccination is, in spite of its virtues, not in widespread use, primarily for two reasons. One reason is the inconveniences associated with the traditional vaccination programme that comprises repeated vaccinations i.a. injections over a several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing.

Conventional specific allergy vaccination is therefore carried out using multiple subcutaneous immunizations applied over an extended time period. The course is divided in two phases, the up dosing and the maintenance phase. In the up dosing phase increasing doses are applied, typically over a 16-week period, starting with minute doses. When the recommended maintenance dose is reached, this dose is applied for the maintenance phase, typically with injections every six weeks. Following each injection the patient must remain under medical attendance for 30 minutes due to the risk of anaphylactic side reactions, which in principle although extremely rare could be life-threatening. In addition, the clinic should be equipped to support emergency treatment. There is no doubt that a vaccine based on a different route of administration would eliminate or reduce the risk for allergic side reactions inherent in the current subcutaneous based vaccine as well as would facilitate a more widespread use, possibly even enabling self vaccination at home.

Attempts to improve vaccines for specific allergy vaccination have been performed for over 30 years and include multifarious approaches. Several approaches have addressed the allergen itself through modification of the IgE reactivity. Others have addressed the route of administration.

The immune system is accessible through the oral cavity and sublingual administration of allergens is a known route of administration. Administration may be carried out by placing the vaccine formulation under the tongue and allowing it to remain there for a short period of time, e.g. 30 to 60 seconds.

Conventionally allergy vaccine using the oromucosal route consists of the up to daily dosing of a solution of the allergen. In comparison, the therapeutic (accumulated) maintenance doses given exceeded the maintenance of the comparable subcutaneous dose by a factor 5-500.

There exists a need for an improved biological vehicle capable of presenting selected proteinaceous compounds (e.g. allergens or antigens) for the manufacture of vaccines.

### SUMMARY OF THE INVENTION

The present invention is directed to a pharmaceutical composition for use as a medicament comprising a biological vehicle surface-displaying one or more heterologous proteinaceous compound comprising:
a) cells of one or more non-pathogenic bacterial strain, and
b) one or more proteinaceous compound covalently bound by means of a bifunctional cross-linker to an accessible chemical entity on the surface of said cells,
   wherein said cells do not comprise a transgenic nucleic acid molecule encoding said one or more proteinaceous compound, and said bi-functional linker is bonded to an amino group of said cells via a Schiff-base, and said proteinaceous compound and said linker are heterologous in origin to said cells. Preferably, said medicament is for the treatment or prophylactic treatment of an animal or human patient.

In a preferred embodiment, said bifunctional linker is selected from the group consisting of glutaraldehyde, polyazetidine and paraformaldehyde.

Furthermore the biological vehicle of the composition may comprise cells of either a non-genetically modified bacterial strain, or a genetically modified bacterial strain or a combination thereof. In a preferred embodiment the bacterial strain of the composition is a member of a bacterial genus selected from the group consisting Lactococcus, Lactobacillus, Leuconostoc, Group N Streptococcus, Enterococcus, Bifidobacterium, non-pathogenic Staphylococcus and non-pathogenic Bacillus.

In one embodiment the one or more proteinaceous compound is an antigen from an animal or human pathogen, or variant thereof. Alternatively, the one or more proteinaceous compound is either an allergen, an animal or human cancer antigen, or a self-antigen of animal or human origin, or variant thereof.

The composition according to the invention may furthermore comprise a bi-functional linker and/or a spacer compound. The number of molecules of proteinaceous compound bound per cell in the composition, comprising a bi-functional linker or spacer, may range of 1 to about 100,000. In the absence of a spacer, the number of molecules of proteinaceous compound bound per cell is in the range of 1 to about 10,000. The composition may furthermore be comprised in an encapsulated formulation.

The composition may be used as a medicament. In particular, the composition may be used for the manufacture of a medicament for the prevention and/or treatment of a disease selected from the group consisting of: infectious disease, cancer, allergy, and autoimmune disease in an animal or human patient. Thus, the composition of the invention may be used in the prevention and/or treatment of a disease or allergy of an animal or human patient, whereby the patient is administered an effective dose of the composition.

The invention further provides a method for the preparation of the pharmaceutical composition of the invention, comprising a biological vehicle surface-displaying one or more heterologous proteinaceous compound, comprising the steps: preparing a mixture comprising: i) cells of one or more bacterial strain, and ii) one or more heterologous proteinaceous compound, and iii) a heterologous bifunctional cross-linker, and incubating said mixture to form said biological vehicle in which said bi-functional linker is bonded to an amino group of said cells via a Schiff-base, and separating said biological vehicle from said mixture, wherein said cells do not comprise a transgenic nucleic acid molecule encoding said one or more proteinaceous compound. In a preferred embodiment, said mixture is incubated at a temperature of below 0° C., preferably at a temperature of between −1° C. and −30° C., most preferably at −20° C.

According to the method of the invention, the biological vehicle may comprise cells of either a non-genetically modified bacterial strain, or a genetically modified bacterial strain. Furthermore the invention is preferably practiced with a bacterial strain that is a member of a bacterial genus selected from the group consisting Lactococcus, Lactobacillus, Leuconostoc, Group N Streptococcus, Enterococcus, Bifidobacterium, non-pathogenic Staphylococcus and non-pathogenic Bacillus.

According to the method of the invention, the one or more proteinaceous compound may be an antigen, or variant thereof, from an animal or human pathogen. In an alternative embodiment, the one or more proteinaceous compound may be an allergen or variant thereof; or the one or more compound may be an animal or human cancer antigen and variant thereof or self-antigen and variant thereof.

In a further embodiment of the method of the invention, the mixture may further comprise a bifunctional linker, and/or a spacer compound. The method may further comprise the step of encapsulating the composition comprising the biological vehicle.

### DESCRIPTION OF THE INVENTION

### I. Brief Description of the Drawings

FIG. 1. Chemical cross-linking to Lactobacillus using 1 μg/ml or 2 μg/ml β-galactosidase. The amount of β-galactosidase activity detected in the cell fraction or supernatant fraction of a cross-linking reaction mixture comprising 10¹⁰ cells.

FIG. 2. Chemical cross-linking of arabinose isomerase to Lactobacillus. The amount of arabinose isomerase activity detected in the cell fraction or supernatant fraction of a cross-linking reaction mixture comprising 10¹⁰ cells. Surface cross-linked enzyme is depicted with triangular symbols and total amount of enzyme with squared symbols.

FIG. 3. Chemical cross-linking of β-galactosidase to Lactobacillus using chitosan as enhancer molecule.

FIG. 4. Chemical cross-linking of Betv1 protein to Lactobacillus cells using glutaraldehyde as described in Example 11.

Panel A shows phase-contrast pictures of pellet material that have been cross-linked using glutaraldehyde. Panel B shows cells from a mixture of Betv1 protein and Lactobacillus cells, where no glutaraldehyde was added. The right hand illustration of each panel shows selected cells from the material analyzed in the left hand illustration, viewed at a higher magnification.

FIG. 5. Surface distribution of Betv1 cross-linked to Lactobacillus cells using glutaraldehyde.

Panel A and B show pictures of cells in pellet material prepared as described in Example 12. Panel A shows cells derived from a cross-linking reaction in which Betv1 protein was present; whereas B shows cells derived from a negative control reaction in which Betv1 protein was omitted. Detection of the Betv1 protein was performed using a primary anti-Betv1 from rabbit and a secondary Cy-3 labelled anti-rabbit antibody as described in Example 12. The pictures to the left are phase contrast images, and the pictures to the right are fluorescent images (filter limits for excitation and emission light were 545-575 nm and 610-680 nm respectively) of the same cells using identical settings of both microscope and camera.

FIG. 6. Spleen cell proliferation after SLIT treatment, immunization and subsequent in vitro re-stimulation. Four groups of mice received once a day for three weeks the following: BetV-Lb: Vaccine conjugates containing BetV1 coupled to L. acidophilus X37; Lb: Untreated L. acidophilus X37; BetV1 2.5 μg: Purified BetV1 protein 2.5 μg per day; BetV1 5 μg: Purified BetV1 protein 5 μg per day, Buffer: negative control group receiving buffer.

FIG. 7. In vitro dendritic cell stimulation using untreated lactobacillus, LacS conjugated lactobacillus or LacS protein alone.

LX37: Untreated L. acidophilus X37; LX37+lacS+glut: Surface coupled B-galactosidase to lactobacillus using glutaraldehyde; LacS: Protein LacS alone.

### II. Definition of Terms

Allergen: Are antigens that elicit a hypersensitivity or allergic reaction

Antigen: Any proteinaceous substance capable of inducing an immune response.

Antigen variant: Any antigen, where the amino acid composition has been changed from the natural antigen.

### API: Active Pharmaceutical Ingredient(s)

Cross-linker: A chemical reagent that contain two reactive groups thereby providing the means of covalently linking two target groups. In homo-bifunctional cross-linkers, the reactive groups are identical forming a covalent bond between similar groups. In hetero-bifunctional cross-linkers, the reactive groups have dissimilar chemistry allowing formation of cross-links between unlike functional groups. Heterologous bifunctional cross-linker is defined as a chemical reagent that has a different origin from (i.e. is not native) to the cell to which it is linked.

### DC: Dendritic Cell

### FDA: Food and Drug Administration

GM: genetically modified
GMO: genetically modified organism
GLA: glutaraldehyde

Heterologous proteinaceous compound is defined as a protein-containing compound that has a different origin from (i.e. is not native to) the cell to which it is surface bound or cross-linked by a covalent or non-covalent bond. M9 buffer: Aqueous solution comprising 0.6% Na₂HPO₄, 0.3% KH₂PO₄, 0.5%, NaCl, 0.025% MgSO₄.

MRS: Medium suitable for cultivation of Lactobacillus

### ONPG: Ortho-Nitrophenyl-β-D-Galactopyranoside

PCR: polymerase chain reaction
Spacer: A molecule with multiple reactive groups that enhances the cross linking reaction. Used as a bridge between the cells surface and the target protein.
Target protein: The proteinaceous compound (to be) displayed on the bacterial cell surface
Transgenic nucleic acid molecule: a nucleic acid molecule that is introduced and stably integrated into the genome (comprising both plasmid, episomal and chromosomal DNA) of a host organism, wherein said DNA comprises a protein coding sequence, and wherein said transgenic nucleic acid molecule is not found in the host organism in nature, but is introduced into the host cell by means of genetic modification techniques.
Room temperature: Between 15-25° C. preferably 18° C.

### III. Detailed Description

The present invention provides a biological vehicle characterized by the surface display of one or more proteinaceous compound, whose properties have particular application in the areas of vaccine delivery, whole-cell bioabsorbents, biofilters, microbiocatalysts and diagnostic tools. The invention lies in the recognition that a therapeutically-effective, safe, and publicly-acceptable vaccine should comprise the following components and properties:
a) a biological non-pathogenic vehicle, preferably capable of locating and attaching temporarily to immuno competent cells in the mucosa of an animal or human (patient),
b) wherein said vehicle provides surface display of one or more heterologous antigen, capable of presentation to immuno-competent cells leading to a specific immune response, and
c) is capable of stimulating—as an adjuvant or immune modulator—the immune cells and thereby the entire immune system and preferably inducing the host cells of said patient to secrete the desired cytokines and
d) wherein the vaccine, comprising a vehicle with one or more surface displayed heterologous antigen, is cheap and simple to produce, and avoiding the need to synthesize complex linkers e.g. bacterial wall murein precursors.

A non-pathogenic bacterium provides the properties of a) and c), whereby specific proteins located on the surface of these bacteria allow them to locate and attach to target cells in the mucosa, and by bacterium—cell cross-talk initiate various responses e.g. cytokine and mucin production (Christensen H. R. et al. 2002, J Immunology 168:171-8, Mack D. R. et al. 2003 Gut 52:827-33) The localisation of bacterial cells to the mucosa may be mediated by mannose-sensitive binding to mammalian cells as described by Adlerberth I. et al., 1996 Appl Environ Microbiol 7:2244-51. Accordingly, the present invention employs non-pathogenic bacterial strains whose surface components are still present, and can thereby support the effective presentation of surface located antigens. The present invention fulfils the requirements of b) by providing a non-pathogenic bacterial cell to which one or more heterologous proteinaceous compounds are surface-bound. The heterologous compound may be affinity bound or adsorbed to the surface of the bacterial cell, or covalently bound employing a coupling agent. Proteinaceous compounds isolated from natural sources or synthesized chemically or produced using recombinant DNA technology may be coupled to the surface of the bacterium of the invention. The heterologous proteinaceous compound that is bound to and displayed on the surface of the bacterium of the invention is not limited to a compound that can be synthesized and secreted by the bacterial cell itself. Said heterologous proteinaceous compound may comprise a post-translational modification whose synthesis relies on catalytic steps not found in the bacterium of the invention. Herein lies one the significant advantages of the invention, in that the heterologous proteinaceous compound displayed on the bacterial surface may be a compound whose composition and structure may be tailored for a specific use, without being limited to a compound that lies within the biosynthetic capacity of the bacterial cell on which it is displayed. The method of the invention can provide a densely packed surface display of proteinaceous compound(s), which serves to enhance their immunogenic properties during antigen presentation. Since the amount of surface-bound proteinaceous compound in a given bacterial sample of the invention can be determined with accuracy, this facilitates the precise control of antigen dose as a therapeutic preparation, which is another significant advantage of the invention.

In contrast to known technologies, based on surface-display of heterologous antigenic proteins by GM bacteria, the non-pathogenic bacterial strain in one embodiment of the present invention is not classified as genetically modified since the heterologous surface-displayed proteinaceous compounds are not recombinantly expressed by the cells themselves. In an alternative embodiment the non-pathogenic bacterial strain, to which one or more heterologous proteinaceous compounds are bound is itself genetically modified.

A non-pathogenic bacterial strain suitable for practicing the present invention includes a Gram-positive bacterial strain, preferably selected from a species from the group of bacterial genera consisting of Lactococcus, Lactobacillus, Leuconostoc, Group N Streptococcus, Enterococcus, Bifidobacterium, non-pathogenic Staphylococcus, non-pathogenic Bacillus. More preferably the non-pathogenic bacterial strain is selected from a species selected from the group of bacterial genera consisting of Lactococcus, Lactobacillus, Leuconostoc, Group N Streptococcus, Enterococcus, Bifidobacterium non-pathogenic Staphylococcus. Even more preferably the non-pathogenic bacterial strain is selected from a species selected from the group of bacterial genera consisting of Lactobacillus and Bifidobacterium.

More specifically the preferred non-pathogenic bacterial strain is selected from a species selected from the group of bacterial species consisting of: Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp delbrueckii, Lactobacillus delbrueckii subsp. lactis, Lactobacillus durianus, Lactobacillus equi, Lactobacillus farciminis, Lactobacillus ferintoshensis, Lactobacillus fermentum, Lactobacillus formicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme, Lactobacillus zeae, Bifidobacterium adolescentis, Bifidobacterium aerophilum, Bifidobacterium angulatum, Bifidobacterium animalis, Bifidobacterium asteroides, Bifidobacterium bifidum, Bifidobacterium boum, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum subsp. longum, Bifidobacterium longum subsp. infantis, Bifidobacterium longum subsp. suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, Bifidobacterium urinalis.

The one or more proteinaceous compound bound to and displayed on the surface of the non-pathogenic bacterium may be selected from a wide variety of compounds, where the protein may further comprise a carbohydrate, lipid or other post-translationally added modifications. Preferably the compound is a substituted, meaning post-translationally modified, or un-substituted protein or peptide, wherein said compound is capable of inducing the development of a humoral or cellular response in animals or humans e.g. antigen, allergen, allergoid, peptide, protein, hapten, glycoprotein, peptide nucleic acid (PNAs, a sort of synthetic genetic mimic), and viral or bacterial material as well as analogues or derivatives thereof. Such modification can be made by chemical modification or synthetic modification, e.g. by PEGylation (PEG=polyethylene glycol), biotinylation, deamination, maleination, substitution of one or more amino acids, by cross-linking, by glycosylation, or by other recombinant or synthetic technology. The term is also intended to include natural-occurring mutations, isoforms and retroinverse analogues.

More preferably said compound is capable of inducing the development of specific antibodies and/or a specific T-cell response in animals or humans. Alternatively said compound is capable of inducing the development of a cytotoxic T-cell response in animals or humans, or the compound is capable of inducing the development of an allergic response. Furthermore the compound may be capable of reacting with pre-existing antibodies or T-cells, or is a compound capable of binding to the IgE antibody on mast cells or mediating a type I allergic response in a previously sensitised mammal. In a preferred embodiment the proteinaceous compound is capable of inducing the development of immunity against one or more infectious agent(s) or allergen(s) in an animal or a human. Alternatively, the proteinaceous compound is capable of inducing the development of immunity against autoimmune diseases in animals or humans. In a further embodiment the proteinaceous compound or variants thereof is one that operates as cancer antigens in animals or humans.

The proteinaceous compound inducing development of immunity in an animal or human may originate from, or be a variant thereof, one or more of the following sources: bacteria, virus, fungi, protozoan and prions for example selected from the following group:

### Antigen Sources

Poxyiridae, Herpesviridae, Adenoviridae, Parvoviridae, Papovaviridae, Hepadnaviridae, Picornaviridae, Caliciviridae, Reoviridae, Togaviridae, Flaviviridae, Arenaviridae, Retroviridae, Bunyaviridae, Orthomyxoviridae, Paramyxoviridae, Rhabdoviridae, Arboviruses, Oncoviruses, Unclassified virus e.g. selected Hepatitis viruses, Astrovirus and Torovirus, Bacillus, Mycobacterium, Plasmodium, Prions (e.g. causing Creutzfeldt-Jakob disease or variants), Cholera, Shigella, Escherichia, Salmonella, Corynebacterium, Borrelia, Haemophilus, Onchocerca, Bordetella, Pneumococcus, Schistosoma, Clostridium, Chlamydia, Streptococcus, Staphylococcus, Campylobacter, Legionella, Toxoplasmose, Listeria, Vibrio, Nocardia, Clostridium, Neisseria, Candida, Trichomonas, Gardnerella, Treponema, Haemophilus, Klebsiella, Enterobacter, Proteus, Pseudomonas, Serratia, Leptospira, Epidermophyton, Microsporum, Trichophyton, Acremonium, Aspergillus, Candida, Fusarium, Scopulariopsis, Onychocola, Scytalidium, Histoplasma, Cryptococcus, Blastomyces, Coccidioides, Paracoccidioides Zygomycetes, Sporothrix, Bordetella, Brucella, Pasteurella, Rickettsia, Bartonella, Yersinia, Giardia, Rhodococcus, Yersinia and Toxoplasma.

The proteinaceous compound for treatment or alleviation of allergy or therapeutic or prophylactic allergy vaccination may originate from one or more of the following sources:

### Allergen Sources

The term “allergen” refers to any naturally occurring protein or mixtures of proteins that have been reported to induce allergic, i.e. IgE mediated reactions upon their repeated exposure to an individual. Allergens, for the purpose of the present invention may be derived from plants, pets, farm animals, insects, arachnids and food, including pollen from birch and taxonomically related trees, Japanese cedar trees, olive trees, ragweed, weeds, or grasses, stinging, insects, mosquitoes/midges, cockroaches, dust mites, indoor fungi, outdoor molds, cattle, cats, dogs, horses, rodents, peanuts, nuts, fruits, milk, soy, wheat, egg, fish and shellfish. In particular, allergens suitably may be an inhalation allergen originating i.a. from trees, grasses, herbs, fungi, house dust mites, storage mites, cockroaches and animal hair and dandruff. Important pollen allergens from trees, grasses and herbs are such originating from the taxonomic orders of Fagales, Oleales and Pinales including i.a. birch (Betula), alder (Alnus), hazel (Corylus), hornbeam (Carpinus) and olive (Olea), the order of Poales including i.a. grasses of the genera Lolium, Phleum, Poa, Cynodon, Dactylis and Secale, the orders of Asterales and Rosales, Urticales including i.a. herbs of the genera Ambrosia, Artemisia and Parietaria. Important inhalation allergens from fungi are i.a. such originating from the genera Alternaria and Cladosporium. Other important inhalation allergens are those from house dust mites of the genus Dermatophagoides and mites an storage mites of the genus Blomia, Euroglyphus and Lepidoglyphus, those from cockroaches and those from mammals such as cat, dog, horse and rodents such as mice, rats, guinea pigs and rabbits. Further, recombinant allergens according to the invention may be venom allergens including such originating from stinging or biting insects such as those from the taxonomic order of Hymenoptera including bees (superfamily Apidae), wasps (superfamily Vespidea), and ants (superfamily Formicoidae).

Specific allergen components include e.g. Bet v 1 (B. verrucosa, birch), Aln g 1 (Alnus glutinosa, alder), Cor a 1 (Corylus avelana, hazel) and Car b 1 (Carpinus betulus, hornbeam) of the Fagales order. Others are Cry j 1 (Pinales), Amb a 1 and 2, Art v 1 (Asterales), Par j 1 (Urticales), Ole e 1 (Oleales), Ave e 1, Cyn d 1, Dac g 1, Fes p 1, Hol l 1, Lol p 1 and 5, Pas n 1, Phl p 1 and 5, Poa p 1, 2 and 5, Sec c 1 and 5, and Sor h 1 (various grass pollens), Alt a 1 and Cla h 1 (fungi), Der f 1 and 2, Der p 1 and 2, Der m 1 (house dust mites, D. farinae, D. pteronyssinus and D. microceras, respectively), Lep d 1 and 2 and Blo t 1 and 2, Eur m 1 and 2, Gly d 1 and 2 (Lepidoglyphus destructor; Blomia Tropicalis and Glyphagus domesticus storage mite and Euroglyphus maynei), Bla g 1 and 2, Per a 1 (cockroaches, Blatella germanica and Periplaneta americana, respectively), Fel d 1 (cat), Can f 1 (dog), Equ c 1, 2 and 3 (horse), Apis m 1 and 2 (honeybee), Ves v 1, 2 and 5, Pol a 1, 2 and 5 (all wasps) and Sol i 1, 2, 3 and 4 (fire ant).

The allergen may be in a form of an allergen extract, an isolated purified allergen and variant or fragments thereof.

The allergen may also be obtained by virtue of recombinant gene expression technology i.e. a recombinant allergen and variants or fragments thereof, or a mutant of and fragments thereof. For example, the recombinant allergen may be a recombinant Betv1, Fel d 1, Phl p 1 or 5, Lol p 1 or 5, Sor h 1, Cyn d 1, Dag g 1 and 5, Der f or p 1 or 2, Amb a 1 and 2, Cry j 1 and 2, Ves v 1, 2 and 5 or Dol m1, 2 and 5, Api m 1 or cockroach Bla g 1 and 2, Per al. Mutant of Bet v 1 whose composition are be modified, and the amino acids in Betv1 that are potentially suitable for substitution comprise amino acids are described in e.g. WO 99/47680, WO02040676, WO03/096869.

The expression “allergen extract” as used therein refers to an extract obtained by extraction of a biological allergen source material as generally described in “Allergenic extracts”, H. Ipsen et al, chapter 20 in Allergy, principle and practise (Ed. S. Manning) 1993, Mosby-Year Book, St. Louis. Such extract may be obtained by aqueous extraction of water soluble material followed by purification steps like filtration to obtain the solution i.e. the extract. The extract may then be subjected to further purification and/or processing like freeze-drying removing substantially all the water. Generally, an allergen extract comprises a mixture of proteins and other molecules. Allergen proteins are often classified as a major allergen, an intermediate allergen, a minor allergen or no classification. An allergen extract generally comprises both major and minor allergens. Major allergens will generally constitute approximately 5-15% of an average allergen extract, more often about 10%. Amounts of allergen extract referred to herein refers to the dry matter content of such allergen extracts.

Preferably the water content of the dry matter does not exceed 10%, more preferably 5% by weight.

Biological allergen source materials may comprise contaminating materials, such as foreign pollen and plant and flower debris for an allergen pollen source material.

The degree of contamination should be minimised. Preferably, the content of contaminants should not exceed 10% (W/W) of the biological source material.

Normally an allergen extract contains at least 10% protein of the dry matter content of the allergen extract as determined in a standard protein assay such as BCA or Lowry and the remainder consists of other “non-protein material,” which may be components such as lipids, carbohydrates, or bound water which originate from the biological allergen source.

An allergen extract may be formulated and stored in form of a freeze-dried material obtainable by freeze-drying a liquid allergen extract at a pressure of below 800 micro bar and for a period of up till 100 hours removing the water. In the field of allergy extracts, there is no international accepted standardisation method. A number of different units of extract strength i.e. bio-potency exist. The methods employed and the units used normally measure the allergen content and biological activity. Examples hereof are SQ-Units (Standardised Quality units), BAU (Biological Allergen Units), BU (biological units), UM (Units of Mass), IU (International Units) and IR (Index of Reactivity). Hence, if extracts of origins other than those disclosed herein are used, they need to be standardised against extract disclosed herein in order to determine their potency in SQ units or any of the above mentioned units. The subject matter is dealt with in “Allergenic extracts”, H. Ipsen et al, chapter 20 in Allergy, principle and practise (Ed. S. Manning) 1993, Mosby-Year Book, St. Louis and Løwenstein H. (1980) Arb Paul Ehrlich Inst 75:122. The bio-potency, i.e. the in vivo allergenic activity, of a given extract depends on a number of factors, the most important being the content of major allergens in the extract, which varies with the composition of the biological source material.

The amount of allergen extract in grams to be used for obtaining a desired bio-potency varies with the type of extract in question, and for a given type of extract the amount of allergen extract varies from one batch to another with the actual bio-potency of the extract.

For a given batch of extract, the amount of allergen extract in grams to be used for obtaining a desired bio-potency may be determined using the following procedure:

a) The bio-potency of various amounts of a reference extract is determined using one or more immunological in vivo tests to establish a relationship between bio-potency and amount of reference extract. Examples of the said immunological in vivo tests are Skin Prick Test (SPT), Conjunctival Provocation Test (CPT), Bronchial Challenge with Allergen (BCA) and various clinical trials in which one or more allergy symptoms is monitored, see for example e.g. Haugaard et al., J Allergy Clin Immunol, Vol. 91, No. 3, pp 709-722, March 1993.
b) On the basis of the established relationship between bio-potency and reference extract, the bio-potency of one or more relevant doses for use in the dosage forms of the invention is selected with due consideration to a balance of the factors of i) the effect of treating or alleviating symptoms of allergy, ii) side effects recorded in the immunological in vivo tests, and iii) the variability of i) and ii) from one individual to another. The balancing is done to obtain a maximal adequate therapeutic effect without experiencing an unacceptable level of side effect. The way of balancing the factors are well known to those skilled in the art

The bio-potency of the one or more relevant doses found may be expressed in any biopotency unit available, such as SQ units, BAU, IR units, IU, cf. above.

c) From the reference extract one or more bio-potency reference standard extracts is prepared and, if used, the bio-potency unit values of the reference standard extracts are calculated on the basis of the bio-potency unit value allocated to the one or more relevant doses, e.g. such a standard for BAU can be obtained from FDA as illustrated below.
d) For the reference standard extracts of each extract type, a number of parameters for evaluating the bio-potency of extracts are selected. Examples of such evaluation parameters are total allergenic activity, the amount of defined major allergens and overall molecular composition of the extract. The total allergenic activity may be measured using an in vitro competitive immunoassay, such as ELISA and MagicLite® luminescence immunoassay (LIA), using a standardised antibody mixture raised against the extract obtained using standard methods, e.g. antibodies raised in mouse or rabbit, or a pool of allergic patients sera. The content of major allergens may e.g. be quantified by rocket immuno-electrophoresis (RIE) and compared to the reference standards. The overall molecular composition may be examined using e.g. crossed immunoelectrophoresis (CIE) and sodium dodecyl sulphate polyacrylamide gel electrophoresis (SDS-PAGE).
e) For a given batch of extract of unknown bio-potency (test extract), the amount of extract to be used for obtaining a desired bio-potency level (effective dose for use in the solid dosage form according to the present invention) may be determined as follows: For each evaluation parameter selected, the test extract is compared with the reference standard extracts using the relevant measurement methods as described above, and on the basis of the measurement results the amount of extract having the desired bio-potency is calculated.

An effective dose of an allergen for allergy treatment or therapeutic or prophylactic allergy vaccination shall mean a dose which when taken once or repeatedly in a monodose or in incremental doses results in, for example, an adaptive immune response and thus serves as means to desensitize allergic patients. Preferably, the term shall mean the amount of allergen in each dosage form necessary to induce an adaptive immune response after repeated administration of said solid dosage forms in accordance with a treatment regimen (over a period ranging from a few applications to at least one daily application over several months). Preferably desensitization includes the alleviation of allergic symptoms upon administration of the dose. Clinical allergy symptoms include rhinitis, conjunctivitis, asthma, urticaria, eczema, which includes reactions in the skin, eyes, nose, upper and lower airways with common symptoms such as redness and itching of eyes and nose, itching and runny nose, coaching, weezing, shortness of breathe, itching, and swelling of tissue.

In an embodiment the dose of an allergen may be an allergen extract content of about 0.15 μg-10 mg/dose, more preferred an allergen extract content of about 0.5 μg-5 mg/dose, more preferably an allergen extract content of about 0.5 μg-3.75 mg/dose, more preferably an allergen extract content of about 2.5 μg-3.75 mg/dose, more preferably an allergen extract content of about 2.5 μg-2.5 mg/dose, more preferably an allergen extract content of about 25 μg-2.5 mg/dose, more preferred about 25 μg-1.25 mg/dose, even more preferred about 25 μg-1 mg/dose, most preferable about 25 μg-0.75 mg/dose.

In a further embodiment an allergen dose has a single allergen content of about 0.015 μg-1 mg/dose, more preferred of about 0.05 μg-500 μg/dose, more preferably of about 0.05 μg-375 μg/dose, more preferably of about 0.25 μg-375 μg/dose, more preferably of about 0.25 μg-250 μg/dosage, more preferably of about 2.5 μg-250 μg/dose, more preferred about 2.5 μg-125 μg/dose, even more preferred about 2.5 μg-100 μg/dose, most preferable about 2.5 μg-75 μg/dose

In a further embodiment, an allergen dose has a single allergen content of about 0.015 μg-1 mg/dosage form, more preferred of about 0.05 μg-500 μg/dosage form, more preferably of about 0.05 μg-375 μg/dosage form, more preferably of about 0.25 μg-375 μg/dosage form, more preferably of about 0.25 μg-250 μg/dosage form, more preferably of about 2.5 μg-250 μg/dosage form, more preferred about 2.5 μg-125 μg/dosage form, even more preferred about 2.5 μg-100 μg/dosage form, most preferable about 2.5 μg-75 μg/dosage form.

### Surface-Coupling of a Protein to the Biological Vehicle of the Invention

The one or more proteinaceous compounds are bound to the surface of the non-pathogenic bacterium by either non-covalent or covalent bonds. The invention further discloses a method for binding said one or more proteinaceous compound to a non-pathogenic bacterial cell employing a chemical cross-linking agent, capable of joining two or more molecules together by a covalent bond. In general, chemical cross-linking reagents contain reactive ends to specific functional groups most often amines or sulfhydryls on proteins or other molecules. Examples of a cross-linking agent suitable for performing the present invention include gluteraldehyde, polyazetidine and paraformaldehyde. The use of the bifunctional cross-linker gluteraldehyde for the chemical cross-linkage of the protein β-galactosidase to the cell surface of Lactobacillus plantarum is disclosed in Examples 1, 2, 6, and 7.

Alternatively a covalent bond between the one or more proteinaceous compounds and the surface of the non-pathogenic bacterium is enzymatically catalysed employing a catalytic agent selected from transferases e.g. transglutaminase, (said enzymes being classified under the Enzyme Classification number E.C. 2 in accordance with the recommendations (1992) of the International Union of Biochemistry and Molecular Biology), oxidoreductases e.g. laccase or horse radish peroxidase (said enzymes being classified under the Enzyme Classification number E.C. 1), peptide ligases (said enzymes being classified under the Enzymé Classification number E.C. 6) or hydrolases e.g. transpeptidase, carboxypeptidase or endopeptidase (said enzymes being classified under the Enzyme Classification number E.C. 3). The use of the transglutaminase for the covalent linkage of the protein β-galactosidase to the cell surface of Lactobacillus plantarum is disclosed in Example 5.

Alternatively the one or more proteinaceous compounds may be non-covalently bound to the surface of the non-pathogenic bacterium by weaker non-specific bonds, as exemplified in Example 4.

The reaction conditions for the chemical or catalytic cross-linkage, or binding of the one or more proteinaceous compounds to the surface of the non-pathogenic bacterium may be modulated in order to vary the number of molecules of proteinaceous compound bound per cell, for example by varying the ratio of cell and protein to be coupled, the pH, the ionic strength of the buffer, and the temperature of the reaction mixture. Thus, in one embodiment of the present invention, the cross-linkage reaction is performed at low temperature, preferably a temperature of below 0° C., more preferably between −1° C. and −20° C., for example −20° C., where a low temperature has surprisingly been shown to result in a higher number of proteinaceous molecules to be covalently bound to the surface of a bacterial cell (see Examples 9-12).

The number of molecules of bound proteinaceous compound may also be enhanced by including a spacer molecule in the reactions mixture, as disclosed in Example 3 and 8. One of the particular advantages of the present invention relates to the number and density of proteinaceous compound molecules that may be bound and displayed on the surface of the bacterial cell of the invention. As illustrated in Examples 1 and 3, proteinaceous compound molecules may be cross-linked directly to a chemical entity on the bacterial surface or indirectly through a multivalent spacer, whereby the number of bound molecules is not limited to the number of native protein molecules that a bacterial cell may have attached to its cell surface in vivo.

The bond by which the heterologous proteinaceous compound is cross-linked to the surface of the non-pathogenic bacterial cell of the invention, is a covalent bond between an accessible chemical entity at the surface of the bacterial cell, on the one hand, and a terminal or internal substituent of the proteinaceous compound on the other hand. The cross-link between the heterologous proteinaceous compound and said accessible entity may further comprise a heterologous bifunctional linker, whereby said cross-linker is an integrated component of the cross-linked product. The bacterial cell of the invention has an outer surface, comprising a wall, that is chemically accessible and to which the heterologous proteinaceous compound may be cross-linked. More specifically, a chemically accessible entity at the surface on the bacterial cell of the invention is a component of the bacterial cell envelope comprising a cell wall or outer cell membrane, wherein said entity is directly exposed to compounds present in the external environment of the cell.

In one embodiment of the present invention, wherein a heterologous proteinaceous compound is non-covalently bound to the surface of a bacterial cell, the number of molecules of compound bound per bacterial cell is at least 100.

### Preparation and Testing of a Vaccine of the Invention:

The present invention provides a method for preparing non-pathogenic bacteria with one or more surface bound proteinaceous compounds, which may be employed in the manufacture of a vaccine and further tested and therapeutically used in an animal or a human, as exemplified in Example 17. In a first step, non-pathogenic bacteria with a proteinaceous compound (e.g. antigen) covalently bound to the cell surface are manufactured by the chemical cross-linking technology as described in the examples e.g. Example 1 or 9. Alternatively, conjugates of bacteria and antigen are manufactured using cross-linking enzymes as described in Example 5 or using non-specific binding as described in Example 4. Thus, by way of example, bacterial cells are produced with surface bound proteinaceous compounds, where the bound compounds are specific antigens of the human pathogenic Mycobacterium tuberculosis or influenza virus, or surface antigens of the animal pathogen E. coli. The bacterial cells having surface bound E. coli antigens are used in the manufacture of a veterinary vaccine for use in livestock swine, in particular to prevent or treat diarrhea in piglets. The steps in the manufacture of a vaccine comprising bacterial cells with specific surface bound antigens for use in the treatment of illnesses caused by Mycobacterium tuberculosis, influenza virus or veterinary E. coli are similar and are described below.

1. Selection of Non-Pathogenic Bacterial Strain for Surface Bound Antigen Presentation

A number of bacterial strains, which have been described to transiently colonize the recipient host, are selected for further analysis. The strains are analysed in the in vitro dendritic cell model as described by Christensen H. R. et al. 2002, J Immunology 168:171-8, and in Example 18. The preferred strain is one that is characterized by the induction of inflammatory cytokines including IL1, IL2, IL6, IL12, TNFα, and/or TGFβ.

2. Antigen Production for Surface Bound Presentation on Non-Pathogenic Bacteria

A M. tuberculosis antigen, for example ESAT6 (Sørensen A. L. et al. 1999 Infect Immun. 63:1710-17), is produced recombinantly in Lactococcus lactis using an expression system, e.g. the P170 Expression system (Madsen S. et al. 1999 Mol. Microbiol. 32:75-87). The gene encoding the antigen is inserted into an expression vector, e.g. pAMJ297, and transformed into a L. lactis strain, which is subsequently cultivated in growth medium in a fermentor as previously described (Madsen S. et al. 1999 supra). The antigen is synthesized and secreted by the transformed L. lactis cells during fermentation. The supernatant is then separated from the L. lactis cell culture using for instance cross-flow filtration. The M. tuberculosis antigen present in the supernatant is purified using traditional protein purification methods, including for example gel filtration. The purified antigen is dissolved in an appropriate buffer e.g. M9, as described in Example 1. An influenza virus antigen is produced either by recombinant gene expression, or by purification of the antigen from intact virus that has been cultivated in eggs as described in Tree J. A. et al. 2001 Vaccine 19(25-26): 3444-50.

3. Production of Non-Pathogenic Bacterial Cells for Use in Surface Bound Antigen Presentation on Bacteria

The strain selected in step 1, is cultivated in a growth medium, which is a complex media e.g. MRS (Oxoid) in the case of preparing a vaccine for animal experimentation and veterinary use. A growth medium, solely based on synthetic components, is used for strain cultivation when preparing a vaccine for human use, due to the risk of infectious agents such as virus and prions that may be present in growth medium components of animal origin. Furthermore, the selected strain growth medium is one that meets the safety guidelines for therapeutic human use issued by for instance the FDA. Following cultivation in a fermentor, the bacterial cells are separated from the growth medium by for instance centrifugation or cross-flow filtration. The bacterial cells are resuspended in fresh growth medium or an appropriate buffer such as M9 buffer. These cells can be stored at −80° C. for at least a year, following addition of an equal volume of 50% autoclaved glycerol.

4. Cross Linking Reaction and Formulation

The bacteria produced in step 3, and the antigen produced in step 2, are surface-bound using one or more of the methods described in Examples 1, 3-12. The resulting non-pathogenic bacteria with surface bound antigens are evaluated in the following tests:

The amount of antigen surface-coupled to the bacterial cell(s) is determined using immuno-detection techniques e.g. ELISA test employing fluorescent-labelled antibodies specific for the bound antigen, or Western blot analysis of extracts of the bacterial cells employing antibodies specific to the bound antigen. In addition, the distribution of antigens on the bacterial surface is analysed using the same antibodies in a microscope-based analysis. The cells containing surface-coupled antigens are suspended in an appropriate buffer e.g. M9 buffer and stored in glycerol at −80° C. as described in step 3. The cells may be used per se. However, the vaccine may also be encapsulated using novel or well known methods. The encapsulation must ensure that the vaccine keeps the original properties during storage and during transit in hostile environments such as gastric juice. Further, the encapsulation shall ensure that the vaccine is released at the desired mucosal location. Various encapsulation methods are described and commercially available both for preserving live or dead microorganisms see for instance http://www.encapdrugdelivery.com (Encap Drug Delivery, UK)

5. Test of Product in an Animal Model

The test vaccine, comprising bacteria with surface-coupled antigens prepared and formulated according to step 4, is divided into aliquots comprising from about 10⁸ to about 10¹¹ cells. Four animal groups, each comprising 10 mice, are vaccinated with either the test vaccine or a control vaccine, as follows: two groups receive the test vaccine in different doses; one group receives a control vaccine comprising the bacterial cells without surface coupled antigen; and one group receives the purified antigen. The vaccine is administered orally or nasally or by nasojejunal tube. The vaccine schedule is as follows. Doses are given at days No.: 1, 2, 3, 14, 15, 16, 42, 43 and 44. Blood and mucosa samples are taken each week starting at day No. 0, where the first blood and mucosa samples constitute the pre-immune sera. The final blood and mucosa samples are taken at day No. 63, and the mice are sacrificed prior to the removal of the spleen and optionally the lymph nodes. The blood and mucosa are analysed for antigen-specific antibodies using standard techniques e.g. ELISA technique. The spleen is analysed for the presence of antigen-specific cytotoxic T-cells using for instance a chrome release assay.

The useful test vaccine according to the foregoing animal vaccination trial exhibits the following properties:
no detectable antigen-specific antibodies or antigen-specific cytotoxic T-cell response in mice treated with a control vaccine comprising the bacterial cells of the invention without surface coupled antigen, and
the levels of antigen-specific antibodies and/or antigen-specific cytotoxic T-cell responses in mice treated with the test vaccine, at least at the higher dose, is greater than the levels detected in mice treated with purified antigen, wherein the difference is statistically significant.

About one molecule of antigen can be coupled per cell by adjusting the concentration of antigen. One dose comprising a single cell with one surface-coupled antigen-molecule defines the lower dose limit. Optimisation of the cross-linking conditions (Example 13) is expected to result in at least 10,000 surface-coupled molecules pr. cell. If the cross-linkage reaction cross-links one molecule of target protein per cell, then one dose of 10¹² cells will contain 83 ng cross-linked target protein. Hence, an efficient chemical cross linkage according to the present invention will cross-link 10,000 molecules of target protein per cell providing about 1 mg of cross-linked protein per dose of 10¹² cells. The use of a bifunctional linker or spacer, more preferably a combination thereof, allows a further increase in the number of molecules of target protein that can be bound to a single cell, to about 100,000. The number of cells in a single dose could also be optimised, thereby increasing the total amount of antigen in single dose. The number of cells and the number of surface-coupled molecules defines the upper dose limit.

### Formulation and Administration of a Vaccine of the Invention:

One embodiment of the invention provides a pharmaceutical composition for the manufacture of a medicament comprising a biological vehicle surface-displaying one or more heterologous proteinaceous compound including: a) cells of one or more non-pathogenic bacterial strain, and b) one or more proteinaceous compound bound by means of a bifunctional cross-linker to an accessible chemical entity on the surface of said cells, wherein said cells do not comprise a transgenic nucleic acid molecule encoding said one or more proteinaceous compound, and said bifunctional linker is covalently bonded to an amino group of said cells via a Schiff-base, and said proteinaceous compound and said linker are heterologous in origin to said cells. Where the surface-displayed proteinaceous compounds are antigens and the biological vehicle are bacteria which are living or dead, the composition may be used per se as a vaccine for any mucosal administration including oromucosal, oral, nasal, sublingual, vaginal or anal administration.

The term “oromucosal administration” refers to a route of administration where the pharmaceutical composition of the invention is placed under the tongue or anywhere else in the oral cavity to allow the active ingredient to come in contact with the mucosa of the oral cavity or the pharynx of the patient in order to obtain a local or systemic effect of the active ingredient. An example of an oromucosal administration route is sublingual administration.

The term “sublingual administration” refers to a route of administration, where a dosage form is placed underneath the tongue in order to obtain a local or systemic effect of the active ingredient. For this use, the vaccine is formulated as either a solution, or a crystallized-, dried or freeze dried-substance together with appropriate materials that preserve the original properties of the vaccine and provide an optimal shelf life. However, the vaccine may also be encapsulated using novel or well-known methods. The encapsulation must ensure that the vaccine keeps the original properties during storage and during transit in hostile environments such as gastric juice. Further, the encapsulation shall ensure that the vaccine is released at the desired mucosal location. Various encapsulation methods are described and commercially available both for preserving live or dead microorganisms see for instance http://www.encapdrugdelivery.com.

The average amount of surface-coupled antigens on each microbial cell and the number of non-pathogenic bacteria in each vaccination dose may be calculated according to the method described in Example 13.

Further to mucosal administration, dermal or subcutaneous administration may be advantageous to vaccinate against or treat selected diseases. Also parenteral administration may be useful for vaccination against or treatment of selected diseases such as cancers. Presentation of the non-pathogenic bacteria containing surface-coupled antigens to tumor, dendritic or other mammal cells ex vivo may be useful prior to transplantation or re-transplantation.

The formulated vaccine may be administered in numerous forms such as fluids, aerosols, powders, crystals and tablets. The formulated vaccine may also contain active substances that adjust the activity of the vaccine or provide additional properties. The active substances could be complex or simple immuno-modulatory compounds such as interleukins or other active pharmaceutical ingredients (API). The API's may be one or more novel or well-known drugs that either enhance the therapeutic effect of the vaccine or derive useful properties from the vaccine when administered simultaneously.

### Identification of the Appropriate Strain to be a Component of Each Specific Vaccine and Pre-Analysis of the Vaccine of the Invention:

A vaccine usually consists of an adjuvant and the specific vaccine components. The role of the adjuvant is to stimulate the immune system and thereby enhance the effects of the specific pathogen or antigens. An important property of the microbial cells in the vaccine is to serve as a mucosal adjuvant and/or as a complex component that directs the immune system to respond in a desired manner in addition to responding to the specific antigen(s). In some aspects the appropriate immune response to a vaccine may be humoral while in other aspects it may be cellular or a combination of both. Further, the response may be polarized into so called Th1 or Th2 responses or polarized towards an inflammatory response or anti-inflammatory response or induce tolerance. By employing the appropriate strain the immune polarization towards the bound protein can be controlled.

The immune system of the mucosa is part of the entire immune system and, consequently, immune responses in the mucosa are reflected in the entire body. It consists of an integrated network of tissues, lymphoid and non-lymphoid cells and effector molecules such as antibodies and cytokines. The interaction between antigen-presenting cells, T lymphocytes and cytokines is the key to providing the correct specific immune response. The meeting between cells of the immune systems, and an infecting agent or antigen, results in the production of interleukins. The interleukins are mediator molecules that instruct the remaining immune system as to how to behave towards the infecting agents or antigen. Essentially, interleukins are divided into two classes that direct either a pro-inflammatory or an anti-inflammatory immune response. However, a huge number of sub-classes must be present since more than 20 interleukins are known and each response to different infections results in different levels of each interleukin.

Methods have been established ex vivo to analyse the immune response to various bacteria (Christensen H. R. et al. 2002, J Immunology 168:171-8). The methods are based on dendritic cells (DCs) that are recognized as the key modulators of the immune system. DCs develop into mature immuno-competent cells when they encounter foreign cells or antigens. During the encounter, the DCs secrete cytokines both to perform a self-stimulation and to stimulate other cells of the immune system. In the ex vivo methods the cytokines from DCs are measured both qualitatively and quantitatively following exposure to inactivated or live microorganisms. Different microorganisms have been tested using such methods and the results show significant variation between different bacteria including variation between members of the same genus and even the same species. Therefore, the DC methods are useful for identifying bacterial candidates for a given vaccine since suitable candidates are those that direct the desired immune response as indicated by the cytokine release profile. The candidates should also direct the desired immune response while simultaneously presenting the specific antigens. Accordingly, bacterial candidates with foreign antigens coupled to their cell surface may also be tested using the DC method, as illustrated in Example 18.

In the future, more sophisticated methods are expected to be developed in order to improve the distinction between the different immune responses and to closely mimic the situation in vivo. Such methods will be useful to identify more precisely the right candidates for specific vaccines.

### Animal Experiments and Application of the Vaccine of the Invention for Veterinary Purposes:

A vaccine may be designed and manufactured by for instance using the methods described in the disclosed examples. The vaccine may contain components to be useful as a vaccine against a pathogen for instance selected from the group of infectious agents and allergen listed under Antigen sources and Allergen source respectively. It may also contain components to be useful as a vaccine against other diseases for instance selected from the group of infectious disease, cancer, allergy and autoimmune disease.

The vaccine may be tested in animal experiments using a formulation method chosen from the examples. The test animals may be of any animal species. The vaccine may be fed to the animals or mixed with the drinking water. The vaccine may also be administered vaginally or anally or the vaccine may be administered directly to the small intestine through devices that surpass the stomach and the gastric juice for instance by using a nasojejunal tube. The vaccine may be administered by spraying the animal directly in the mouth-nose or gill region or simply by spraying out the vaccine in the animal stables. The vaccine may also be added to the water where fish and other water borne animal live. The administration may be performed once or followed up regularly to ensure a vaccination boost and maintenance of immune memory. The experiment may be performed with vaccination or placebo treatment before and/or after challenge with pathogens or induction of an illness that resembles the disease in question. The endpoints of the experiment may include but are not limited to analysis of the number of surviving animals and healthy animals versus dead or ill animal. The severity of illness among the surviving animals may also be an important parameter. Biopsies from the treated animals and specific antibody titers may also indicate the effect of the vaccination. Cells from the Biopsies may also be tested in immuno-assays including specific responses to the antigen(s) in question.

The animal experiments may be designed to identify:

the right non-pathogenic bacterium of a number of candidates for use in the particular vaccine,

the ideal dose of non-pathogenic bacterium with the ideal average number of surface-coupled antigens,

the optimal route of administration,

substances that enhance the effect of the vaccine, and

the vaccination frequency and the vaccination period.

The results of the animal experiments will be useful for setting up the regime for vaccination of domestic animals, pets, farm animals, herds, livestock or wild animals. Hereafter, the vaccine will be useful for veterinary purposes.

In some aspects, the animal results may be useful for the design of human vaccine trials. Also, animal experiments may be useful for testing human vaccine in pre-clinical trials.

### Human Trials and Application of the Vaccine for Human Purposes

As mentioned in the previous example, a vaccine may be designed and manufactured by for instance using the methods described above. The vaccine may comprise components useful as a vaccine against a pathogen or any allergy listed under above Allergen sources. It may also contain components to be useful as a vaccine against cancers or auto-immune diseases or other disease selected for instance from the group listed under above Antigen sources. The vaccine may be tested in clinical trials following the completion of pre-clinical trials as described for a veterinary vaccine. The formulation method may be chosen from any one described in the examples. Healthy and/or ill persons may be included in the clinical trial. The vaccine may be taken as tablet(s), part of the food or as a drink. The vaccine may be administered sublingually or sprayed in the mouth and nose region. The vaccine may also be administered vaginally or anally or the vaccine may be administered directly to the small intestine through devices that surpass the stomach and the gastric juice for instance by using a nasojejunal tube. The administration may be performed once or followed up regularly to ensure a vaccination boost and maintenance of immune memory. The vaccination may be performed with the vaccine and/or placebo and may be using a randomized double-blinded approach. The endpoints of the experiment may include but are not limited to analysis of the number of survivors and the healthy persons versus dead or ill persons. The severity of illness among the survivors may also be an important parameter. Biopsies from the treated survivors, the treated healthy and ill persons may be used for analysing the results from the clinical trials. Also, the specific antibody titers may indicate the effect of the vaccination. Cells from the biopsies may also be tested in immuno-assays including specific responses to the antigen(s) in question.

The clinical trials may be designed to identify and select:

a non-pathogenic bacterium from a number of candidates for use in a particular vaccine,

the optimal dose of non-pathogenic bacterium with the optimal average number of surface-coupled antigens,

the optimal route of administration,

substances that enhance the effect of the vaccine, and

the vaccination frequency and the vaccination period.

The results from the clinical trials are useful for setting up the regime for vaccination of humans, at which point the vaccine will be useful for human vaccination purposes.

DCs exposed ex vivo to a vaccine containing surface-coupled antigens related to diseases such as cancers may also be useful for the treatment of the diseases. The ex vivo exposure may be performed prior to re-transplantation of DCs to the patient. Further, a vaccine in which the same type of antigens have been surface-coupled to live or dead non-pathogenic bacteria may be used parenterally for providing the proper adjuvant effect to vaccines such as cancer vaccines.

The method of the invention may also be useful for producing an efficient and easy-to-administer vaccine against a pathogen in a bio-terror attack. It is known that for instance existing licensed anthrax vaccines must be administered parenterally and require multiple doses to induce protective immunity (Flick-Smith H. C. et al., 2002, Infection and Immunity, 70:2022). This requires trained personnel and is not the optimal route for stimulating a mucosal immune response. Also, the administration is not ideal for vaccination of a large number of persons within a very short time.

### III Examples

### Example 1

### Chemical Cross-Linkage of β-Galactosidase to Lactobacillus by Glutaraldehyde

This example demonstrates the chemical cross-linkage of the protein β-galactosidase from Sulfolobus solfataricus (Pisani F. M. et al. 1990 Eur J Biochem., 187:321-8) to the cell surface of Lactobacillus plantarum UP1 using the bifunctional cross-linking reagent, glutaraldehyde (GLA), which is a five-carbon dialdehyde. GLA acts as a cross-linker by forming a Schiff-base (—H═N—) with the amino groups of proteins. Thus GLA mediated cross-linkage of β-galactosidase to the bacterial surface is expected to occur between lysine or arginine residues present in the β-galactosidase protein and accessible lysine or arginine residues on, or near, the cell surface of the bacterium.

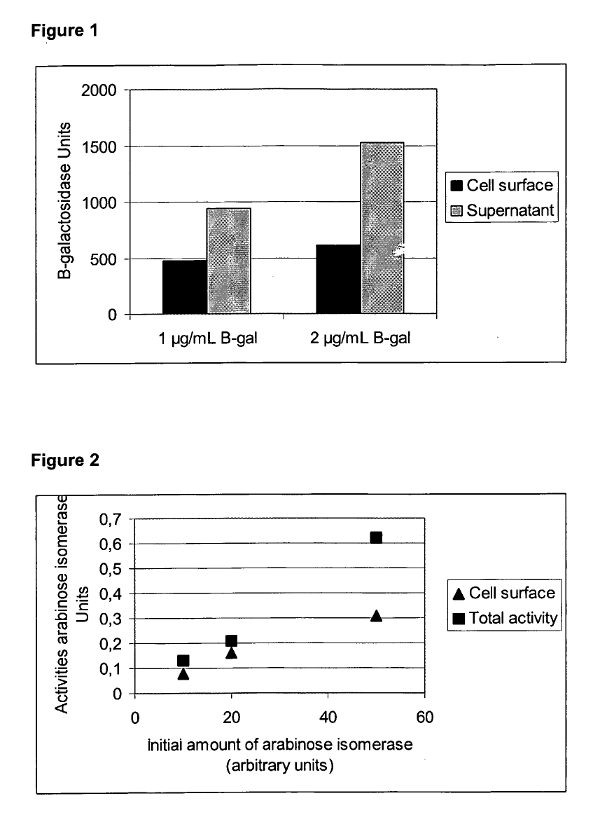

The β-galactosidase for cross-linking studies was obtained by recombinant expression in Escherichia coli, using the pET-3a vector system (Invitrogen, CA). Briefly, the lacS gene, encoding β-galactosidase, was amplified by standard PCR techniques from S. solfataricus genomic DNA, cloned into the pGET-3a vector, and transformed into E. coli. β-Galactosidase was expressed intracellularly, and the E. coli cells were then lysed. The β-galactosidase, released into the lysate was partially purified by thermo precipitation, by heating the lysate to 80° C. for 30 minutes. L. plantarum UP1 was grown in MRS (Oxoid, Hampshire UK) for 24 h at 30° C. without aeration. The cells were harvested by centrifugation and re-suspended in M9 buffer (0.6% Na₂HPO₄, 0.3% KH₂PO₄, 0.5% NaCl, 0.025% MgSO₄) and adjusted to a cell density of 10¹⁰ cells per mL. A fixed amount of L. plantarum cells (1010) was incubated with different amounts of the β-galactosidase and 0.2% GLA (Sigma-Aldrich, St. Lois Mo.) for 50 min at room temperature. The viability of GLA treated cells was tested by spreading the cell mixture on to MRS agar. No viable colonies were produced indicating that the GLA-treatment had killed the majority of the cells. Subsequent to the GLA treatment, the cell mixture was subjected to centrifugation to give a cell fraction and a supernatant. The isolated cells were thoroughly washed twice in M9 buffer. In order to monitor GLA-mediated cross-linkage of 9-galactosidase to the L. plantarum cells, the distribution of β-galactosidase between the supernatant and cell fractions was determined by assaying β-galactosidase enzyme activity employing the ONPG procedure described by Sambrook J et al., 1989, Molecular Cloning, A Laboratory Manual, Cold Spring Harbour Press, Plainview, N.Y.). Due to the repeated M9 wash of the cells, the amount of unbound β-galactosidase in the cell fraction was negligible. Therefore, the enzyme activity measured in the cell fraction is taken to correspond to the amount of enzyme, which is bound to the surface of the L. plantarum cells. Cross-linkage using 1 μg/mL and 2 μg/mL β-galactosidase and 10¹⁰ cell per mL resulted in binding of 33% and 40%, respectively, of the total amount of enzyme (based on detectable units of enzyme activity) to the surface of the cells (FIG. 1), whereas less than 5% was surface bound in the control reaction without GLA (data not shown). The cell fractions, obtained after GLA cross-linkage with either 1 μg/mL or 2 μg/mL β-galactosidase, had enzyme activities at 480 U and 610 U, which corresponds to 600 and 800 molecules of β-galactosidase per cell, respectively. In conclusion, the example demonstrates that GLA is able to cross-link an enzymatically active protein to the surface of a Lactobacillus cell. The enzyme cross-linked to the cell surface retains its enzymatic activity, indicating that GLA-mediated protein cross-linking to the cell surface does not compromise the functionality of the protein.

### Example 2

### Chemical Cross Linkage of Arabinose Isomerase to Lactobacillus by Glutaraldehyde

To demonstrate that chemical cross-linking of proteins to the cell surface of a bacterium is not limited to β-galactosidase, we show the cross-linkage of the enzyme arabinose isomerase, from the thermophilic Thermoanaerobacter mathrani, to a bacterial cell. Arabinose isomerase converts D-galactose to D-tagatose and was obtained by recombinant intracellular expression in E. coli as described by Jørgensen and co-workers (Jørgensen F et al. 2004, Appl Microbiol Biotechnol 64:816-22). After growth and expression in recombinant E. coli, the cells were lysed using a French press. This lysed mixture was centrifuged and the supernatant comprising arabinose isomerase was used for the following cross-linking experiment. Lactobacillus plantarum UP1 was grown and washed as described in Example 1. The washed cells (10¹⁰ cells) were incubated with different amounts of lysate containing the arabinose isomerase and with GLA at a final concentration of 0.1%. The cross-linking reaction was performed at 37° C. for 60 minutes. Thereafter, the cells were harvested and washed as described in Example 1. Enzyme activities in both the cell fraction and the supernatant were analyzed as previously described (Jorgensen F et al. 2004, Appl Microbiol Biotechnol 64:816-22). FIG. 2 shows that arabinose isomerase is cross-linked to the cell surface of Lactobacillus in a concentration dependent matter. Since the arabinose isomerase enzyme was not inhibited by the GLA treatment (data not shown), the total detectable catalytic activity in the cell and supernatant fractions (FIG. 2) corresponds to the amount of enzyme added to the washed L. plantarum cells prior to GLA treatment. FIG. 2 shows that more than 50% of the arabinose isomerase was bound to the surface of the L. plantarum cells under all three cross-linking conditions, based on comparing the arabinose isomerase activity in the cell fractions with the total amount of enzyme activity in the cell and supernatant fractions combined. In conclusion, this example shows that GLA can efficiently cross-link active arabinose isomerase to the surface of a Lactobacillus cell.

### Example 3

### Chitosan, as Spacer Molecule, Enhances Levels of β-Galactosidase Cross-Linked to Lactobacillus by Glutaraldehyde

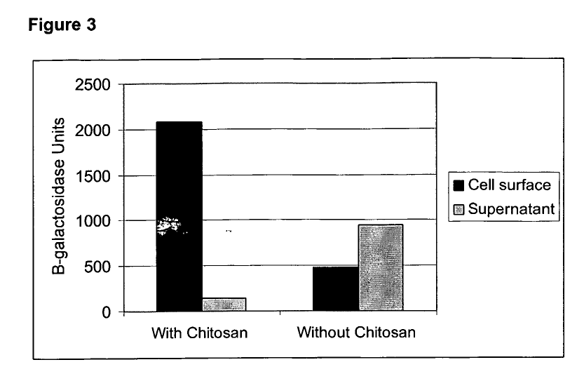

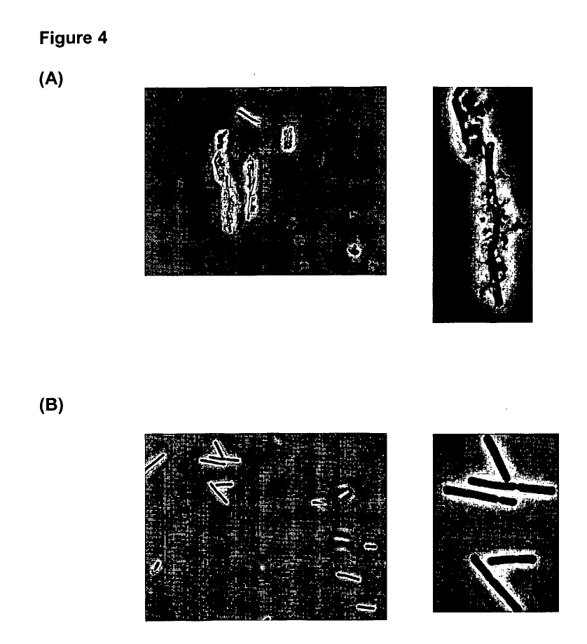

Chitosan is a naturally occurring molecule, containing multiple reactive groups, which can be used as spacer molecule to enhance the amount of protein attached to the surface of the bacterial cell by chemical cross-linkage. L. plantarum cells were grown and washed as described in Example 1, and suspended in M9 buffer at a concentration of 10¹⁰ cells per ml, to which 0.5% w/v chitosan 500 kDA (Cognis Deutschland GmbH, Germany), and 0.2% GLA were added, together with either 1 μg/mL or 2 μg/mL β-galactosidase. The effect of chitosan on the cross-linkage of β-galactosidase to cells was compared to a control cross-linking reaction without chitosan. The L. plantarum cells were harvested and washed as described in Example 1 and β-galactosidase catalytic activity of the washed cell fraction and the supernatant of the cross-linking reaction mixture were measured. FIG. 3 demonstrates that chitosan enhances the cross-linkage reaction, since more than 90% of the total β-galactosidase activity was bound to the cell fraction, while only 35% of the β-galactosidase activity was bound to the cell fraction in the absence of chitosan. This example shows that using chitosan as molecular spacer enhances the cross-linking reaction and increases the amount of bound β-galactosidase from 800 molecules per cell to approximately 2000 molecules per cell.

### Example 4

### Non-Covalent Binding of Proteins to the Surface of Lactobacillus

The chemical treatment of Lactobacillus with cross-linking reagent results in non-living cells. This example shows that untreated and live bacteria can bind β-galactosidase in a non-covalent manner. While not bound by theory, the interaction between the cell surface and the protein is either ionic, hydrophobic or between the β-galactosidase and sugar moieties on the cell surface.

Lactobacillus plantarum UP1 was grown and washed as described in Example 1. Cells (10¹⁰) were incubated for 60 minutes at 37° C. with 2 μg/ml β-galactosidase. The cells were centrifuged and washed twice in 500 μl M9 buffer. β-Galactosidase catalytic activity in the washed cell fraction and supernatant from the cross-linking reaction mixture were analysed. In the cell fraction 538 U were detected, whereas 5455 U were measured in the supernatant corresponding to a binding of 9% of the total β-galactosidase to the cell surface. This example shows that Lactobacillus can bind β-galactosidase to the cell surface without the use of cross-linking mediators. However, only 9% of the added β-galactosidase is bound by said non-covalent cross-linking, whereas using GLA reagent, as described in Example 1, and similar amounts of β-galactosidase, resulted in 40% of the added β-galactosidase being bound. The non-covalent binding method is therefore four fold less efficient as compared to covalent binding method using the GLA method. The decrease in bound β-gal (40% to 9%) corresponds to a decrease from 800 to 180 molecules β-galactosidase per cell. The reaction may be optimised to obtain higher amounts of bound protein. Optimisation may be achieved by controlling the pH, the ionic strength of the buffer, the temperature or other parameters.

### Example 5

### Enzymatic Catalysed Cross Linkage of β-Galactosidase to Lactobacillus

The enzyme transglutaminase (TG) is capable of forming inter- and intra-molecular cross-links in and between proteins. Therefore, TG can catalyse the cross-linkage between externally added proteins and amino acids residues in components of the cell wall of bacteria. Through an acyl transfer reaction, TG catalyses cross-links between the γ-carboxyamide groups of peptide- or protein-bound glutamine residues as acyl donors and several primary amines as acyl acceptors, including the ε-amino groups of peptide- or protein-bound lysine residues. This reaction leads to the formation of cross-links in the form of ε-(γ-glutamyl)lysine isopeptides, when protein-bound lysine residues act as acyl acceptors. The reaction is performed by mixing bacterial cells with the target protein (eg. antigens or allergens) and TG, where cross-linking gradually increases with time. The reaction conditions are preferably buffered at pH of between about 6.5 and about 8.0, and comprise ≧10 mM CaCl₂, to optimise the cross-linking reaction. Furthermore, heat treatment at ≧90° C. of the target protein and addition of 20 mM dithiothreitol may enhance cross-linkage. Since TG inhibitor substances in milk proteins have been reported (Böenisch et al. 2004, Jour Food Science 69(8)) heat treatment of the bacterial cells derived from milk may be performed before the cross-linking reaction. Furthermore, the target protein to be cross-linked may be N- or C-terminally extended with amino acids containing multiple reactive residues, which may function as TG substrate and enhance the cross-linking reaction. TG-mediated cross-linkage of a target protein to the surface of bacterial cells is detected by analysing the washed cell fraction and the supernatant for the target protein, either by measuring its functional activity (e.g. catalytic activity) or by immunochemical techniques using antibodies specific for the target protein such as an enzyme, antigen or allergen.

### Example 6

### Cross-Linkage of Beta-Lactoglobulin to Lactobacillus by Use of Glutaraldehyde

Beta-lactoglobulin (BLG) is a whey fraction milk protein causing allergy. The preparation of compositions comprising BLG, capable of eliciting a mucosal immune response after oral administration, may be employed in the treatment of BLG allergy in patients suffering such allergy.

The following components were included in assays performed to demonstrate cross-linkage of BLG to Lactobacillus cells:

Lactobacillus cells: A culture of Lactobacillus plantarum (299v) was grown over night in MRS broth (Flukka 69966) at 30° C. Aliquots of a 1 ml overnight (o/n) culture were centrifuged and the resulting pellets were washed with 1 ml M9 buffer before they were frozen at −20° C. for later use. Before use the frozen pellets were thawed and resuspended in M9 buffer (pellet from 1 ml o/n culture resuspended in 500 ml M9 buffer).
BLG: A 1% solution of BLG (L 6879, Sigma-Aldrich) prepared in sterile distilled water.
Glutaraldehyde (GLA): 25% solution in water of glutaraldehyde (1.04239, Merck).
Glutaraldehyde is a five-carbon dialdehyde, that acts as a cross-linker by forming a Schiff base (>C═N—) with primary amino groups (mostly lysine in the case of proteins).

Samples comprising Lactobacillus cells, BGL protein and glutaraldehyde, in the volumes indicated in Table 1, were mixed and incubated for 60 min at room temperature with periodic mixing.

TABLE 1

| Tube nr. | Cells | BLG | H₂O | GLA |
|---|---|---|---|---|
| 1 | 250 μl | 25 μl | 225 μl | 5 μl |
| 2 | 250 μl | 0 μl | 250 μl | 5 μl |
| 3 | 250 μl | 25 μl | 225 μl | 0 μl |
| 4 | 250 μl | 0 μl | 250 μl | 0 μl |

After 60 minutes incubation, the samples were centrifuged, and the cell-pellets were washed 2 times with 500 μl M9 buffer (M9 buffer: 7.3 gram Na₂HPO₄, 2.9 gram KH₂PO₄ and 2.0 gram NH₄Cl dissolved in water to a total volume of 1 liter).

The amount of cross-linked BLG in the pellet was determined using the Bovine Beta-Lactoglobulin ELISA Quantitation Kit (Catalog nr. E10-125, Bethyl Laboratories) with some modifications, employing solutions described by the kit manufacturer. Because the BLG protein to be detected had been cross-linked to the surface of Lactobacillus cells, the ELISA kit procedure was modified, allowing use of whole cells in suspension for the antibody-based measurement. Briefly, detection was performed using a rabbit anti-BGL antibody conjugated to HRP (horseradish peroxidase). The pellet was resuspended in 100 μl blocking solution, mixed with 100 μl detection solution (1 ml dilution buffer+0.1 μl HRP antibody), and incubated for 60 min at room temperature with regular mixing. The cells were then centrifuged and washed 3 times with 200 μl wash solution before being resuspended in 100 μl dilution buffer. Finally a TMB (Tetramethylbenzidine) color reaction was performed by adding 100 μl TMB reagent (T 0440, Sigma Aldrich) to 50 μl volumes of serially-diluted material in a microtiter plate. The microtiter plate was incubated for 5-30 min at room temperature before the reaction was stopped by addition of 100 μl 2M H₂SO₄ and the absorbance determined at 450 nm in a plate reader. A standard curve linking OD₄₅₀ values and BGL concentrations was included in the microtiter plate setup as described by the kit manual.

TABLE 2

| | OD₄₂₀ for a | OD₄₂₀ for a | Calculated |
|---|---|---|---|
| Tube nr. | 4 × dilution | 8 × dilution | BLG |
| 1 | 0.974 | 0.526 | 65 ng/ml |
| 2 | 0.386 | 0.217 | 19 ng/ml |
| 3 | 0.621 | 0.353 | 38 ng/ml |
| 4 | 0.298 | 0.157 | 11 ng/ml |

Calculated BLG in Table 2 is the protein concentration derived from the measured OD₄₂₀ values by use of the standard curve. A background value 450 nm absorbance is detected in the absence of BGL addition to the reaction tube, which probably reflects non-specific binding of the HRP antibody. Non-specific adhesion of BLG protein to Lactobacillus cells probably explains the levels of BLG in sample 3, where glutaraldehyde is omitted.

### Example 7

### Cross-Linkage of S. Aureus Nuclease to Lactobacillus by Use of Glutaraldehyde

Staphylococcus aureus is an important bacterial pathogen, where treatment is especially difficult due to the emergence of multi-resistant bacterial strains. A candidate vaccine component is the secreted nuclease (Nuc) from Staphylococcus aureus, which have been produced by heterologous protein expression in Lactococcus lactis (Poquet I. et al. 1998 J. Bact., 180:1904-1912).

The following components were included in assays performed to demonstrate cross-linkage of Nuc protein to Lactobacillus cells:

Lactobacillus cells: A culture of Lactobacillus acidophilus (X37) grown and prepared as described in Example 6.
Nuc protein (A): Purified Nuc protein (1 mg/ml, Calbiochem)
Nuc protein (B): Recombinant Nuc protein (187 μg/ml) produced in Lactococcus lactis.

M9 buffer and glutaraldehyde (GLA) are as described in Example 6. Samples comprising Lactobacillus cells, M9 buffer, Nuc protein and glutaraldehyde, in the volumes indicated in Table 3, were mixed and incubated for 60 min at room temperature with periodic mixing.

TABLE 3

| Tube nr. | X37 cells | Nuc | M9 buffer | GLA |
|---|---|---|---|---|
| 1 | 100 μl | 20 μl | 0 μl | 2 μl |
| 2 | 50 μl | 10 μl | 60 μl | 2 μl |
| 3 | 25 μl | 5 μl | 90 μl | 2 μl |
| 4 | 100 μl | 20 μl | 0 μl | 2 μl |
| 5 | 50 μl | 10 μl | 60 μl | 2 μl |
| 6 | 25 μl | 5 μl | 90 μl | 2 μl |

After 60 minutes incubation, the samples were centrifuged, and the pellets were washed 2 times with 1 ml M9 buffer. The Nuc protein concentrations in the pellet material were determined using an antibody-based assay with a primary Nuc antibody (rabbit anti-Nuc antibodies) and a secondary AP-linked anti-rabbit antibody (phosphatase-labeled, affinity-purified antibody to rabbit IgG produced in goat, Catalog nr. 075-1506, KPL), employing solutions described in Example 1. Briefly, the pellet was resuspended in 100 μl blocking buffer, mixed with 100 μl detection solution (1 ml dilution buffer+1 μl Nuc antibody), and incubated for 60 min at room temperature with regular mixing. Then the cells were centrifuged and washed 2 times with 500 μl wash solution before being resuspended in 100 μl dilution buffer. Next 100 μl detection solution (4 ml dilution buffer+1 μl AP antibody) was added, and the sample was incubated for 60 min at room temperature with regular mixing. The sample was then centrifuged, washed 3 times with 500 μl wash solution and resuspended in 100 μl dilution buffer. Finally an AP (alkaline phosphatase) color reaction was performed by adding 100 μl Blue Phos Microwell Phosphatase Substrate System (50-88-02, KPL) to 50 μl volumes of serially diluted material in a microtiter plate. The microtiter plate was incubated for 10-30 min at room temperature before the reaction was stopped by addition of 100 μl 2.5% EDTA and the absorbance determined at 595 nm in a plate reader. A standard curve linking OD₅₉₅ values and Nuc concentrations was generated separately using the same experimental setup.

TABLE 4

| | OD₅₉₅ for a | OD₅₉₅ for a | Calculated |
|---|---|---|---|
| Tube nr. | 4 × dilution | 8 × dilution | Nuc |
| 1 | 0.089 | 0.074 | 13 μg/ml |
| 2 | 0.074 | 0.061 | 8 μg/ml |
| 3 | 0.061 | 0.054 | 5 μg/ml |
| 4 | 0.131 | 0.091 | 22 μg/ml |
| 5 | 0.112 | 0.079 | 18 μg/ml |
| 6 | 0.091 | 0.070 | 13 μg/ml |

Calculated Nuc values given in Table 4 is the protein concentration derived from the measured OD₅₉₅ values by use of the standard curve after background value subtraction. Cross-linkage of Nuc protein to Lactobacillus acidophilus cells is demonstrated and the amount of cross-linked Nuc is proportional to the amount of Nuc protein and cells present in the assay.

### Example 8

### Cross-Linkage of lacS Beta-Galactosidase to Lactobacillus by Use of Glutaraldehyde and Chitosan

Chitosan is a naturally occurring molecule that contains multiple amino-groups, which can participate in glutaraldehyde cross-linking reactions. So-called spacer molecules are often added to cross-linking reaction mixtures in order to improve the outcome.

The following components were employed in assays performed to demonstrate cross-linkage of beta-galactosidase to Lactobacillus cells via chitosan:

Beta-galactosidase was obtained by recombinant expression of the lacs gene from Sulfolobus solfataricus (Pisani F. M. et al. 1990 Eur J. Biochem., 187:321-8) in Escherichia coli using the pET-3a vector system (Invitrogen, CA). Briefly, a PCR fragment encoding the beta-galactosidase was amplified using standard PCR techniques, cloned into the pET-3a vector, and transformed into E. coli for intracellular expression of the beta-galactosidase. A preparation of the expressed lacs protein was obtained by lysis of the E. coli cells followed by a partial purification (thermo-precipitation), where the lysate was heated to 80° C. for 30 min and centrifuged to remove most of the other proteins in the lysate (the lacS beta-galactosidase is a thermostable enzyme). The concentration of lacS beta-galactosidase protein was estimated to be circa 400 μg/ml based on detectable protein in a Coomassie-stained gel.

Lactobacillus cells: A culture of Lactobacillus plantarum (299v) grown and prepared as described in Example 6.

A 1% chitosan solution was prepared by mixing 7.3 mg chitosan (500 kDa) (Cognis Deutchland GmbH, Germany), 730 μl H₂O and 20 μl 2N HCl. M9 buffer and glutaraldehyde (GLA) are as described in Example 6. Samples, comprising Lactobacillus cells, M9 buffer and chitosan in the volumes indicated in Table 5, were mixed and incubated for 5 min at room temperature. lacS beta-galactosidase and glutaraldehyde were then added in the volumes indicated in Table 5, and the samples mixed for 15 min at room temperature.

TABLE 5

| Tube nr. | Cells | M9 buffer | Chitosan | lacS | GLA |
|---|---|---|---|---|---|
| 1 | 50 μl | 50 μl | 2.5 μl | 20 μl | 2 μl |
| 2 | 50 μl | 50 μl | 5 μl | 20 μl | 2 μl |
| 3 | 50 μl | 50 μl | 10 μl | 20 μl | 2 μl |
| 4 | 50 μl | 50 μl | 20 μl | 20 μl | 2 μl |

After 15 min incubation, the samples were centrifuged, and the pellets were resuspended in 100 μM9 buffer. A standard assay for beta-galactosidase enzyme activity employing the ONPG procedure (Sambrook J et al., 1989, Molecular Cloning, A Laboratory Manual, Cold Spring Harbour Press, Plainview, N.Y.) was performed on pellet and supernatant solutions at 65° C. in order to determine the lacS protein distribution. The resulting relative amounts of beta-galactosidase enzyme are shown in Table 6, where the tabulated values are calculated as enzyme activity (units per ml) times volume of the solution (ml).

TABLE 6

| Tube nr. | Pellet | Supernatant | Cross-linked |
|---|---|---|---|
| 1 | 151 | 2427 | 6% |
| 2 | 371 | 2172 | 14% |
| 3 | 653 | 1790 | 25% |
| 4 | 1305 | 1347 | 50% |

Cross-linked is the ratio between the pellet and the total lacs beta-galactosidase activities measured.

This example shows that the inclusion of chitosan as a spacer, significantly increases the yield of cross-linked material.

### Example 9

### Cold Cross-Linkage of Azocasein to Lactobacillus by Glutaraldehyde

We have surprisingly discovered, that cross-linkage by glutaraldehyde can be performed using a freezing protocol (cold cross-linkage), and that high yields of cross-linked protein can be obtained using this protocol.

The following components were included in assays performed to demonstrate cross-linkage of azocasein to Lactobacillus cells:

Azocasein: is well-known as a general protease substrate. It consists of casein conjugated to an azo-dye, which can be used for quantitative spectroscopic measurements. For the cross-linking experiment a 1% solution (10 mg/ml) of azocasein (A 2765, Sigma-Aldrich) dissolved in sterile distilled water was prepared.

Lactobacillus cells: A culture of Lactobacillus plantarum (299v) grown and prepared as described in Example 6.

M9 buffer and glutaraldehyde (GLA) are as described in Example 6.

Samples, comprising Lactobacillus cells, azocasein, M9-buffer and glutaraldehyde in the volumes indicated in Table 7, were mixed and immediately frozen using liquid nitrogen, before being placed in a −20° C. freezer, where the samples were kept for 3 days.

TABLE 7

| 299v cells | Azocasein | M9 buffer | GLA | Recovery |
|---|---|---|---|---|
| 100 μl | 50 μl | 50 μl | 0 μl | 90% |
| 100 μl | 50 μl | 50 μl | 2 μl | 5% |
| 0 μl | 50 μl | 150 μl | 2 μl | 10% |
| 100 μl | 100 μl | 0 μl | 0 μl | 78% |
| 100 μl | 100 μl | 0 μl | 2 μl | 6% |
| 0 μl | 100 μl | 100 μl | 2 μl | 5% |

To evaluate cross-linking, the samples were thawed, centrifuged and the azo-casein content of the supernatants were measured by detecting the azo-group absorbance at 420 nm. Recovery values, given in Table 7, are calculated as the percentage of azo-stain remaining in the supernatant relative to a control (pure azocasein in water similar to initial concentration). Both 2.5 mg/ml and 5.0 mg/ml solutions of azocasein are found to be efficiently crosslinked using 0.25% glutaraldehyde according to the above cold protocol. Centrifugation yielded a firm pellet, which was impossible to resuspend. This indicates, that a high degree of cross-linking has occurred. Crosslinking results with and without the addition of Lactobacillus cells are very similar. Due to the freezing protocol, where the position of the cells is fixed during the incubation, cold cross-linking results in cells and azo-casein uniformly linked into a conglomerate.

### Example 10

### Cold Cross-Linkage of Beta-Galactosidase to Lactobacillus by Glutaraldehyde

The freezing protocol (cold cross-linkage) for the glutaraldehyde-mediated cross-linkage of proteins to the surface of Lactobacillus also gives higher yields of cross-linked beta-galactosidase (lacs) to Lactobacillus.

The following components were included in assays performed to demonstrate cross-linkage of lacs to Lactobacillus cells using the freezing protocol:

Lactobacillus cells: A culture of Lactobacillus acidophilus (X37) was grown and prepared as described in Example 6.
lacS was prepared as described in Example 8.
M9 buffer and glutaraldehyde (GLA) were prepared as described in Example 6.

Samples comprising Lactobacillus cells, lacs beta-galactosidase protein solution, M9-buffer and glutaraldehyde were mixed in volumes indicated in Table 8 and immediately frozen using liquid nitrogen, before being placed in a −20° C. freezer, where the samples were kept for 3 days.

TABLE 8

| Tube nr. | X37 cells | lacS | M9 buffer | GLA |
|---|---|---|---|---|
| 1 | 100 μl | 0 μl | 50 μl | 2 μl |
| 2 | 100 μl | 10 μl | 40 μl | 2 μl |
| 3 | 100 μl | 20 μl | 30 μl | 2 μl |
| 4 | 100 μl | 50 μl | 0 μl | 2 μl |
| 5 | 100 μl | 50 μl | 0 μl | 0 μl |
| 6 | 0 μl | 10 μl | 140 μl | 2 μl |
| 7 | 0 μl | 20 μl | 130 μl | 2 μl |
| 8 | 0 μl | 50 μl | 100 μl | 2 μl |

To evaluate cross-linking the samples were thawed, centrifuged and the pellet was washed once with 200 μM9 buffer. Finally, the pellet was resuspended in 100 μl M9 buffer.

Beta-galactosidase activity was determined as described in Example 7 using an ONPG assay at 65° C. The resulting relative amounts of beta-galactosidase activity are shown in Table 9. The tabulated values are calculated as enzyme activity (units per ml) times volume of the solution (ml).

TABLE 9

| Tube | | | | | Cross- |
|---|---|---|---|---|---|
| nr. | Pellet | Supernatant | Wash | Recovery | linked |
| 1 | 18 | 22 | 13 | — | — |
| 2 | 583 | 139 | 8 | 73% | 80% |
| 3 | 1331 | 77 | 4 | 70% | 94% |
| 4 | 3708 | 10 | 46 | 75% | 99% |
| 5 | 3 | 5016 | 4 | 100% | — |
| 6 | 317 | 188 | 4 | 51% | 62% |
| 7 | 1321 | 14 | 4 | 67% | 99% |
| 8 | 2711 | 18 | 27 | 55% | 98% |

Recovery is the fraction of added lacs activity detected after cross-linking in the pellet+supernatant+wash solutions, combined. Glutaraldehyde treatment leads to enzyme inactivation, which most likely explains the observed loss in total recoverable activity. Addition of X37 cells improves the recovery, probably due to more targets being present for the glutaraldehyde reaction.

Cross-linked values are the beta-galactosidase activity of the pellet expressed as a percentage of the total lacS beta-galactosidase activity. In general, most of the added lacs beta-galactosidase protein is cross-linked, and most enzyme activity is found in the pellet fraction, irrespective of whether 299v cells are included or not. For higher protein concentrations, the recovery is close to 100%.

Microscopy of the pellet fractions showed normal X37 single cells for the sample without GLA (Tube nr. 5). When cross-linking was performed without addition of cells (Tubes 6-8) small aggregates were observed, which were similar in size to bacterial cells. Mixed aggregates, that appeared to consist of both cells and protein material, were observed for the experiments where both cells and lacs beta-galactosidase protein were added (Tubes 2-4). Cells incubated without added lacs also displayed small lumps of cross-linked cells (Tube nr. 1).

### Example 11

### Cold Cross-Linkage of Betv1 to Lactobacillus by Glutaraldehyde Compared with Cross-Linkage at Room Temperature

Betv1, the major pollen antigen from birch (Betula verrucosa), is a 17 kd protein (Breiteneder H. et al. 1989 EMBO J., 8:1935-1938), and it is one of the main causes of Type I allergic reactions (allergic bronchial asthma). The following components were included in assays performed to determine the efficiency of cold cross-linkage of Betv1 to Lactobacillus cells:

Purified recombinant Betv1 protein was obtained according to the procedure described by Spangfort et al. (1996) Prot. Exp. Purification, 8, 365-373. Radioactively labelled Betv1 protein was produced using an in vitro protein synthesis system (RTS 100 E. coli HY Kit, Roche Applied Science). Briefly, a PCR fragment containing the Betv1 reading frame was cloned into the pIVEX2.3d vector (Roche) by way of Nde1 and Sal1 restriction sites added to the PCR primers used for its amplification. Purified plasmid DNA from a resulting clone, that by sequence analysis was found to contain an error-free Betv1 coding sequence, was used as the DNA template for the in vitro protein synthesis. Radioactive labeling was performed using L-[³⁵S]methionine (SJ235, Amersham Biosciences) as described by the manufacturer. A centrifugal 30K filter (Ultrafree, Amicon Bioseparations, Millipore) capable of retaining the Betv1 protein was used to wash low molecular weight products away from the in vitro synthesized protein. The ³⁵S-labelled Betv1 protein was purified by this step, giving a single dominant radioactive band on SDS PAGE. Finally the ³⁵S-labelled Betv1 protein was mixed with M9 buffer and non-radioactive carrier Betv1 (40 μg/ml) to produce the mixture used in radioactive labeling experiments (10 μl ³⁵S labelled Betv1 protein, 490 μl M9 buffer, 550 μl carrier Betv1).

Lactobacillus cells: A culture of Lactobacillus acidophilus (X37) was grown and prepared as described in Example 6.
Betv1: 1.32 mg/ml in sodium phosphate buffer containing 50% glycerol.
M9 buffer and glutaraldehyde (GLA) were prepared as described in Example 6.

Lactobacillus cells, Betv1 protein solutions, and glutaraldehyde were mixed in the volumes indicated in Table 10 and immediately frozen using liquid nitrogen, before being placed in a −20° C. freezer.

TABLE 10

| Tube nr. | X37 cells | Betv1 | ³⁵S-Betv1 | GLA |
|---|---|---|---|---|
| 1 | 100 μl | 5 μl | 10 μl | 2 μl |
| 2 | 100 μl | 5 μl | 10 μl | 2 μl |
| 3 | 100 μl | 5 μl | 10 μl | 2 μl |
| 4 | 100 μl | 5 μl | 10 μl | 2 μl |
| 5 | 100 μl | 5 μl | 10 μl | 0 μl |
| 6 | 100 μl | 5 μl | 10 μl | 0 μl |

After 3 days at −20° C., the samples were thawed were centrifuged. The pellets were washed with 200 μM9 buffer and resuspended in 100 μl M9 buffer. Measurement of ³⁵S-radioactivity in the pellet, supernatant and wash solutions were performed by placing 5 μl drops on a non-absorbing paper surface, drying the drops at 50° C. and placing a super sensitive Storage Phosphor Screen (Pachard Instrument Company) over the dried drops in a film cassette. The exposed Phosphor Screen was scanned (Cyclone, Pachard Instrument Company) and the detected light unit (DLU) signals were integrated for circular areas having identical diameters for all analyzed spots.

The results are shown in Table 11 (relative units), where the abulated values are calculated as scanning result (DLU per ml) times solution volume (ml).

TABLE 11

| Tube | | | | | Cross- |
|---|---|---|---|---|---|
| nr. | Pellet | Supernatant | Wash | Recovery | linked |
| 1 | 713 | 5362 | 431 | 79% | 9% |
| 2 | 916 | 7829 | 355 | 110% | 11% |
| 3 | 1339 | 6873 | 542 | 106% | 16% |
| 4 | 508 | 4215 | 371 | 62% | 6% |
| 5 | — | 6282 | — | 76% | — |
| 6 | — | 7146 | — | 87% | — |

Recovery is the fraction of added ³⁵S-Betv1 activity detected after cross-linking in the pellet+supernatant+wash solutions. Cross-linked is the percentage of total ³⁵S-Betv1 activity detected in the pellet. Dilutions of the ³⁵S-Betv1 solution were used to determine the total radioactivity added, and areas of the non-absorbing paper to which samples had not been localized were used for background subtractions from the determined DLU values.

Microscopy of the pellet fractions showed normal X37 single cells for the samples without GLA, whereas GLA cross-linking produced cells in small aggregates decorated with Betv1 protein material (FIG. 4). The majority of the Betv1 protein seen in the pellet fraction was found to be cell associated, while large protein aggregates were, and small aggregates would be have been removed during the repeated steps of cell washing.

Glutaraldehyde is a widely used protein cross-linking agent (Migneault I. et al. 2004 BioTechniques, 37:790-802). In the comparative experiment glutaraldehyde at room temperature was used to cross-link Betv1 to 2 different types of Lactobacillus cells.

The following components were included in assays performed to determine the efficiency of room-temperature cross-linkage of Betv1 to Lactobacillus cells

Lactobacillus cells (A): A culture of Lactobacillus acidophilus (X37) grown and prepared as described in Example 6.
Lactobacillus cells (B): A culture of Lactobacillus rhamnosus (616) grown and prepared as described in Example 6.
Betv1: 1.32 mg/ml in sodium phosphate buffer containing 50% glycerol. ³⁵S-Betv1 was prepared as described above.
M9 buffer and glutaraldehyde (GLA) was prepared as described in Example 6.

Lactobacillus cells, Betv1 protein solutions, and glutaraldehyde were mixed according to the volumes indicated in Table 12 and incubated at room temperature for 60 min with periodic mixing.

TABLE 12

| Tube nr. | Cells | M9 buffer | Betv1 | ³⁵S-Betv1 | GLA |
|---|---|---|---|---|---|
| 1 | 100 μl (A) | 0 μl | 3 μl | 10 μl | 1 μl |
| 2 | 100 μl (A) | 0 μl | 3 μl | 10 μl | 2 μl |
| 3 | 100 μl (A) | 0 μl | 6 μl | 10 μl | 1 μl |
| 4 | 100 μl (A) | 0 μl | 6 μl | 10 μl | 2 μl |
| 5 | 100 μl (B) | 0 μl | 3 μl | 10 μl | 1 μl |
| 6 | 100 μl (B) | 0 μl | 3 μl | 10 μl | 2 μl |
| 7 | 100 μl (B) | 0 μl | 6 μl | 10 μl | 1 μl |
| 8 | 100 μl (B) | 0 μl | 6 μl | 10 μl | 2 μl |
| 9 | 0 μl | 100 μl | 3 μl | 10 μl | 1 μl |
| 10 | 0 μl | 100 μl | 6 μl | 10 μl | 1 μl |

After the room temperature incubation, the samples were centrifuged, and the pellets washed 3 times with 100 μl M9 buffer and resuspended in 50 μl M9 buffer. Measurement of ³⁵S-activity in the pellet, supernatant and wash solutions were performed by placing 5 μl drops of the sample on a non-absorbing paper surface and processed as described above. The results are shown in Table 13 (relative units), and the tabulated values are calculated as scanning result (DLU per ml) times solution volume (ml).

TABLE 13

| | | | | | | Cross- |
|---|---|---|---|---|---|---|
| Tube nr. | Pellet | Supernatant | Wash-1 | Wash-2 | Recovery | linked |
| 1 | 342 | 16278 | 712 | 74 | 78% | 1.5% |
| 2 | 478 | 16837 | 1278 | 74 | 84% | 2.1% |
| 3 | 258 | 15134 | 642 | 58 | 72% | 1.2% |
| 4 | 367 | 13520 | 615 | 79 | 65% | 1.6% |
| 5 | 316 | 21299 | 816 | 79 | 101% | 1.4% |
| 6 | 604 | 11829 | 958 | 33 | 60% | 3.6% |
| 7 | 312 | 15682 | 803 | 59 | 76% | 1.6% |
| 8 | 154 | 10072 | 1347 | 77 | 52% | 0.7% |
| 9 | 38 | 15011 | 529 | 45 | 70% | 0.2% |
| 10 | 28 | 12613 | 787 | 56 | 61% | 0.1% |

The tabulated values for Recovery and Cross-linked were calculated as described above. Pellet material was examined in the microscope, and cells of both Lactobacillus strains were found to produce somewhat larger cell-aggregates as a result of cross-linking, although cell aggregation was reduced, when M9 buffer was used to dilute the cell concentration in a similar experiment (data not shown).

The yield of small aggregates of Betv1 protein cross-linked with Lactobacillus cells was found to be in the 1-3% range when performed at room temperature, while it was increased to an average cross-linking of 11% using the Cold cross-linking procedure, which is a significant and unexpected improvement in the level of cross-linkage.

A Lactobacillus conjugate with surface-coupled Betv1 prepared according to the above two cross-linking procedures may be concentrated 100 fold. A 5 μl aliquot of each concentrate, when administered to mice in a S.L.I.T. type experiment according to Example 15, would give a dose comprising 0.330 mg or 0.075 mg of Betv1 protein attached to Lactobacillus cells prepared according to the cold or room temperature cross-linking respectively.

### Example 12

### Surface Distribution of Betv1 Cross-Linked to Lactobacillus by Glutaraldehyde

The surface distribution of Betv1 cross-linked to Lactobacillus by glutaraldehyde was examined employing antibody-based detection methods. The following volumes were mixed and immediately frozen using liquid nitrogen, before being placed in a −20° C. freezer: 100 μl Lactobacillus acidophilus (X37) cells, 5 μl Betv1 protein and 2 μl GLA. All solutions are as described in Example 8. A negative control, where Betv1 was omitted from the cross-linking mixture, was treated in an identical manner to the other samples of the experiment.

After 3 days at −20° C., the mixture was thawed, centrifuged and the pellet washed with M9 buffer. In order to avoid auto-fluorescence from residual glutaraldehyde, the pellet was first resuspended in 500 μl 40 mM ethanolamine and incubated for 2 hours at room temperature. Each sample was then centrifuged and the pellet then resuspended in 2 ml NaBH₄ solution (1 mg/ml in PBS buffer, pH 8.0) for 10 min at room temperature. Finally, the pellet material was washed 3 times with 500 μM9 buffer.

The presence of Betv1 was visualized by using a rabbit anti-Betv1 antibody (ALK-Abelló A/S) in combination with a secondary Cy-3 labelled anti-rabbit antibody (PA43004, Amersham Biosciences). Briefly, the pellet was resuspended in 500 μl TBS (50 mM Tris, 0.9% NaCl, pH 7.6) together with 1 μl primary antibody (rabbit anti-Betv1) and incubated 60 min at room temperature. The sample was then centrifuged and washed 3 times in 500 μl TBS buffer, and the pellet was resuspended in 500 μl TBS together with 1 μl secondary antibody (Cy-3 anti-rabbit) and incubated for 60 min at room temperature in the dark. Finally, the pellet was washed 3 times with 500 μl PBS, resuspended in 500 μl PBS and analyzed by fluorescence microscopy (Axioskop 2, Zeiss) with a CCD camera (Princeton Instuments), where pictures were produced by use of the MetaMorph software (Universal Imaging Company).

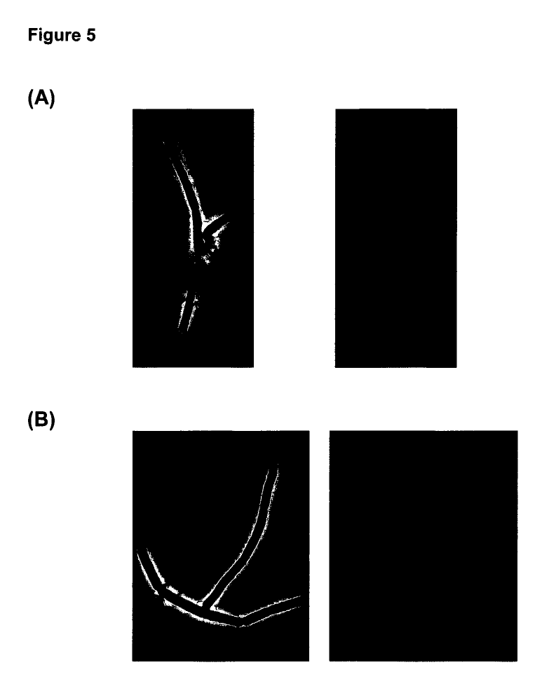

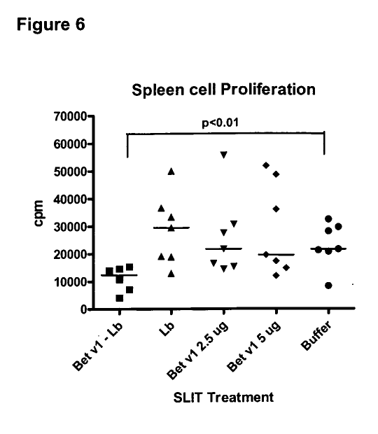

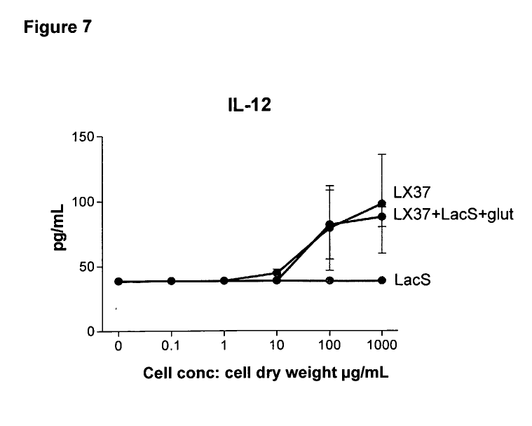

A clear fluorescent signal was localized to the surface of Lactobacillus cells when samples were viewed under the microscope and camera employing identical settings. This demonstrates that Betv1 protein may be cross-linked to the surface of Lactobacillus cells (FIG. 5), and that the cross-linked attached protein (betv1) retains its antibody recognition properties.

### Example 13

### Optimisation and Control of Chemical Cross-Linking of a Proteinaceous Compound to the Surface of Non-Pathogenic Bacteria

The rate of formation of chemical cross-links between a target proteinaceous compound and the cell surface of bacterial cell, and the amount of target protein bound per cell, can be modulated by the cross-linking reaction conditions. For example the incubation time and temperature is set to obtain the required degree of cross-linking. The chemical cross-linker concentration and the target protein to cell ratio is adjusted in the cross-linking reaction to yield a cross-linkage density of about 1 ng to at least about 1 mg of cross-linked target protein per 10¹² cells. Furthermore, the mixing of the reagents during the reaction process is defined to ensure both efficient contact between the reagents, a uniform distribution of the protein on the outer surface of the bacterial cells, and to prevent the formation of cell aggregates. Cross-linking reactions conditions that modulate the abundance, density and distribution of target proteinaceous compound bound to a bacterial cell(s) is analysed by immunochemical methods and microscopy. Alternative bi-functional chemical reagents and alternative spacers for cross-linking may be tested. The disclosed method of the invention allows the production of one or more bacterial cell comprising a controlled amount of cross-linked protein on their outer surface.

### Example 14

### Dosage Estimate of Target Proteinaceous Compound Cross-Linked to Non-Pathogenic Bacteria

The dosage of target proteinaceous compound (eg. enzyme, antigen or allergen) cross-linked to bacterial cells, or formulated as a vaccine, is calculated as follows:

Dosage cross-linked protein=N×M×CFU/A

Where N is the number of cross-linked protein molecules per cell, M is the molecular weight, CFU is the number of colony forming units in one dose and A is Avogadro's number 6.02×1023 mol⁻¹.

In Example 1, the number of surface-coupled β-galactosidase molecules was estimated to about 600-800 molecules per cell, based on the β-galactosidase enzyme activity on the bacterial surface. The estimation assumes that the enzyme activity is conserved after completion of coupling of antigen molecules to the bacterial surface. However, the enzyme activity may be significantly reduced for instance due to the GLA treatment and/or incomplete access of substrate molecules to all of the cross-linked β-galactosidase. Accordingly, the number of surface-coupled molecules may be higher than the estimated 600-800. The number of molecules per cell may be between 1,200 to 2,400 if the enzyme activity is reduced a two-three times by the GLA treatment. The correct number of surface-coupled molecules can be determined precisely, using for instance isotope-labelled antigen or immuno-based techniques.

In Examples 1 and 2, the protocol for coupling the antigen molecules to the bacterial surface was not saturating, since the addition of increasing amounts of antigen in the coupling reaction was shown to increase the amount of surface-coupled antigen (FIGS. 1 and 2). The amount of surface-coupled antigen can be further increased by modulating the cross-linking reaction conditions (including GLA concentration, temperature and/or time of incubation), as detailed in Example 7.

In Example 6, the amount of cross-linked BLG protein was approximately 46 ng/ml. The BGL protein has a molecular weight of around 18,300. Assuming 2×10⁹ cells/ml for a Lactobacillus culture grown over night, this amounts to 4.6 ng BLG protein per 1×10⁹ cells for the reaction volumes used or 151 cross-linked molecules per cell.

In Example 7, the amount of cross-linked Nuc protein was approximately 22 μg/ml for the highest Nuc concentration used. The Nuc protein molecular weight is approximately 18 kd. Assuming 2×10⁹ cells/ml for a Lactobacillus culture grown over night, this amounts to 2.2 μg Nuc protein per 4×10⁸ cells for the reaction volumes used or approximately 184×10³ cross-linked molecules per cell.

In Example 8, the number of cross-linked lacs molecules was found to be between 6 and 50% depending on the amount of chitosan used. The lacs beta-galactosidase is a 57 kd protein. Assuming 2×10⁹ cells/ml for a Lactobacillus culture grown over night, this amounts to from 0.48 to 4.0 μg lacs protein per 2×10⁸ cells or from 25×10³ to 211×10³ cross-linked molecules per cell.

In Example 9, the number of cross-linked azocasein molecules was found to be around 90% of the added material, which amounts to 450 μg per 100 μl cell solution used. Casein is found in several different forms with molecular weights in the 20-25 kd range. Assuming 2×10⁹ cells/ml for a Lactobacillus culture grown over night, this amounts to 450 μg casein per 4×10⁸ cells or approximately 27×10⁶ cross-linked molecules per cell.

In Example 10, the number of cross-linked lacS molecules was found to be in the 80 to 90% range, which amounts to approximately 18 μg per 100 μl cell solution for the highest lacs volume used. Assuming 2×10⁹ cells/ml for a Lactobacillus culture grown over night, this amounts to 18 μg lacs protein per 4×10⁸ cells or approximately 475×10³ cross-linked molecules per cell.

In Example 11, the number of cross-linked Betv1 molecules using the cold procedure was found to be around 10% of the added material, which amounts to 0.66 μg per 100 μl cell solution used. Betv1 is a 17 kd protein. Assuming 2×10⁹ cells/ml for a Lactobacillus culture grown over night, this amounts to 0.66 μg Betv1 protein per 4×10⁸ cells or approximately 58×10³ cross-linked molecules per cell.

Also in Example 11, the number of cross-linked Betv1 molecules using the room temperature procedure was found to be in the 1 to 2% range, which amounts to around 0.2 μg per 100 μl cell solution for the highest Betv1 volume used. Assuming 2×10⁹ cells/ml for a Lactobacillus culture grown over night, this amounts to 0.2 μg Betv1 protein per 4×10⁸ cells or approximately 18×10³ cross-linked molecules per cell.

In Example 1, the target protein (β-galactosidase) cross-linked to the surface of the bacterial cell has a mass of about 50 kDa. Thus, the amount of target protein in one dose containing 10¹² cells and with about 1,000 cross-linked target protein molecules per cell is:

Dosage cross-linked target protein=1,000×50,000g×mol⁻¹×10¹²/6.02×10²³mol⁻¹=83μg

### Example 15

### Use of Lactobacillus Conjugates Containing Surface-Coupled Birch Pollen Allergen BetV1 as a Medicament for the Treatment of Allergy in an Animal Model by Sublingual Administration

### 15.1 Methods:

Animals: Female, 6-10 week-old Balb/cJ mice were breed in-house, and housed in a specific pathogen-free environment under a 12-h light, 12-h dark cycle. All experiments described here were conducted in accordance with Danish legislation.

### 15.2 Manufacture of BetV1 L. Acidophilus X37 Conjugates:

The covalently coupled L. acidophilus X371 BetV1 allergen conjugates was prepared by a repeated glutaraldehyde reaction as described in the following:

L. acidophilus X37 obtained from Bioneer A/S internal strain collection was grown for two days in 250 ml MRS medium at 30° C. without aeration. Cells were harvested and washed in 100 ml M9 buffer and the resulting cell pellet kept in −20° C. until used. The cell pellet was dissolved in 125 ml M9 buffer and portioned as 10 ml in twelve 50 ml Nunc tubes. To this cell suspension and each tube 10 ml M9 buffer, 150 μl 25% glutaraldehyde (1.04239, Merck), and 150 μl BetV1 (with a concentration of 2.56 mg/ml) was mixed and incubated at room temperature for 60 min and frequently mixed. The mixture was centrifuged at 4000 RPM and the supernatant kept and stored at −20° C. for later use. The resulting cell pellet was washed with 10 ml M9 buffer, resuspended in 5 ml M9, pooled, and then centrifuged (4000 RPM) and the resulting cell pellet was dissolved in minimal volumes of M9 buffer. The result was 2.5 ml cell suspension, which was kept over night at −80° C. This cell suspension was subjected to a repeated cross linking reaction using the kept supernatant from the initial cross linking reaction. The cell suspension was portioned as 500 μl in four 50 ml Nunc tubes and to each tube 25 ml BetV1 (kept supernatant), 10 ml acetone, and 50 μl 25% glutaraldehyde was added. The reaction was done at room temperature for 60 min and frequently mixed. The cells were harvested by centrifugation, washed in 10 ml M9 buffer and dissolved in minimal amounts of M9. The result was a 1.5 ml cell suspension that was kept at −80° C. until used as an immune therapy treatment.

### 15.3 Sublingual Immune Therapy (SLIT) Treatment:

Mice were treated 5 days a week for a period of 3 weeks with either a medicament comprising Bet v1 bound to the surface of Lactobacillus Acidophilus X-37 manufactured by the room temperature cross-linkage method described in example 15.2 (2.5 μg Bet v1 and 2.5×10⁹ bacteria per dose); or control compositions comprising a) untreated Lactobacillus Acidophilus X-37 (2.5×10⁹ bacteria per dose), b) Bet v1 having two different concentrations (2.5 and 5.0 μg per dose), or c) buffer. After two weeks of SLIT treatment, the mice were immunized with 10 μg Bet v1 adsorbed to Alum and again after three weeks of treatment. 11 days after the last immunization the mice were sacrificed, the spleen isolated, and spleen cells were restimulated in vitro as described below.

### 15.4 T-Cell Proliferation and Cytokine Production:

Spleen tissue, derived from the treated mice, was teased into single cell suspensions and washed in RPMI-1640 (BioWhittaker, Belgium). Cells were counted and adjusted to 1.67×10⁶ cells/mL in RPMI-1640 containing 50 μg/mL gentamycin (Gibco, UK), 1% Nutridoma (Roche, Germany) and 1.5 mM monothioglycerol (Sigma, USA). 3×10⁵ cells were added to each well of a 96 well flat-bottomed culture plate (Nunc, Denmark) and the cells were stimulated by Bet v1 (0, 5 and 40 μg/mL). The cells were cultured for 6 days at 37° C. and in an atmosphere of 5% CO₂. Proliferation was measured by adding 0.5 μCi of ³H-thymidine to each well for the last 18 hours of the culture period, followed by harvesting the cells on a Tomtec 96 well plate harvester (Tomtec, USA) and counting the incorporated radiolabel using a Wallac Microbeta 1450 Liquid scintillation counter (Wallac, Finland).

### 15.5 Results:

T-cell response was evaluated by measuring the proliferation of spleen cells, isolated from mice subjected to treatment with either Lactobacillus conjugates containing covalently coupled birch pollen allergen BetV1 or control compositions, upon in vitro restimulation with Bet v1. FIG. 6 shows that pre-treatment with the Bet v1 Lactobacillus Acidophilus X-37 conjugates resulted in a significantly reduced spleen cell proliferation, which is indicative of suppression of the allergen reactive T cells and thereby a suppression of the allergic response. This was not the case, when mice were pre-treated with either Lactobacillus Acidophilus X-37, or Bet v1 alone.

### Example 16

### Use of a Staphylococcus aureus Vaccine Based on Lactobacillus Conjugate Comprising Surface-Coupled S. Aureus Nuclease as a Medicament for the Treatment of a Bacterial Infection in an Animal Model

The S. aureus antigen nuclease for use in the present invention can be produced using the L. lactis expression system Madsen et al., 1999 Mol. Microbiol. 32:75-87 and the method described in example 7. A vaccine conjugate is prepared as described in example 7 or using the optimized protocol as described in example 11. The Lactobacillus strain used is the L. acidophilus X37, but others showing a high adjuvant effect are also tested. The adjuvant effect of the different strains is tested in the dendritic cell model described in example 18.

The vaccine conjugate containing surface-coupled nuclease to a selected Lactobacillus strain is tested as an oral vaccine in mice experiments. Naive mice are to be divided in four groups. Group 1 receives orally 300 μL of 10⁸-10¹² bacteria per ml; group 2 receives untreated bacteria at same concentration; group 3 receives nuclease protein alone in a concentration corresponding to that of the conjugates; and group 4 receives phosphate buffer alone. The various treatments are administered orally to the mice once a day for three weeks. Blood samples are be taken and antibodies specific for the nuclease are analyzed using ELISA methods. Furthermore other vaccine schedules and strategies are tested eg. one immunization a week for five weeks or nasal administration is used instead of oral administration.

### Example 17

### The Manufacture of an Allergy Vaccine According the Method of the Invention, and the Administration of the Vaccine in an Animal Model

The optimized chemical cross-linking technology described in Example 11 is used to manufacture a non-pathogenic bacteria with allergen covalently bound to the cell surface. This example focuses on the production of a vaccine with allergens from peanuts or from the milk allergen B-lactoglobulin. The manufacture of the allergy vaccine employs the following steps.

### 17.1. Strain Selection

A number of bacterial strains are analysed in the in vitro dendritic cell model as described in example 18 and by Christensen H. R. et al. 2002, J Immunology 168:171-8. The preferred strain is one that is characterized by significant induction of either a Th1 polarized immune response or induction of tolerance towards the displayed allergen.

### 17.2. Allergen Production

The peanut allergen Ara H2 is produced in E. coli using a gene expression system. The gene encoding the allergen is inserted into a compatible expression vector, pAMJ297, and introduced into a L. lactis strain, which is subsequently cultivated in growth medium in a fermentor as described by Madsen S. et al. 1999 Mol. Microbiol. 32:75-87. The allergen is synthesized and secreted by the recombinant L. lactis cells during fermentative growth. The supernatant is then separated from the cell culture using for instance cross-flow filtration. The recombinant allergen in the supernatant is purified using traditional protein purification methods, for example gel filtration. The resulting allergen is dissolved in an appropriate buffer eg. M9. The B-lactoglobulin allergen is obtained as described in example 1.

### 17.3. Production of Non-Pathogenic Bacterial Cell Biomass

The strain selected in step 1, is cultivated in an appropriate growth medium, employing a complex media, e.g. MRS (Oxoid) for the preparation of a vaccine for animal experiments and veterinary use. A growth medium solely based on synthetic components is employed for strain cultivation for the preparation of a vaccine for human use due to the risk of infectious agents e.g. virus and prions, in growth medium components of animal origin. Growth medium used in preparation of vaccines for human use should also meet safety guidelines issued by for instance the FDA. Following cultivation in a fermentor, the bacterial cells are separated from the growth medium using for instance cross-flow filtration. The bacterial cells are resuspended in fresh growth medium or an appropriate buffer, e.g. M9 buffer. By the addition of an equal volume of 50% autoclaved glycerol the cells can be stored at −80° C. for at least a year.

### 17.4. Cross Linking Reaction and Formulation

The bacteria produced in step 3, and the allergen produced in step 2, are cross-linked using the method described in example 6. The resulting bacteria with surface bound allergen are evaluated in the following tests:

The amount of surface-coupled allergen is determined using immuno-techniques for instance by applying allergen-specific, fluorescent-labelled antibodies in an ELISA test. Alternatively, the amount of surface bound allergen present in an extract of the cells from the cross-linking reaction is determined the method in example 6 using radioactively-labelled allergen. In addition, the distribution of allergens on the bacterial surface is analyzed using the same antibodies in a microscopic analysis. The cells containing surface-coupled allergen are suspended in buffer e.g. M9 buffer and stored in glycerol at −80° C. as described in step 3.
17.5. Test of Bacteria with Surface-Coupled Antigen in an Animal Allergy Model

The cells containing surface-coupled allergen from step 4 comprise a test vaccine that is divided into aliquots containing from 10⁸ to 10¹¹ cells. Four groups, each containing 10 mice, are vaccinated with the test vaccine or a control vaccine according to the following protocol: two groups receive different amounts of test vaccine; one group receives control vaccine comprising the bacterial cells without surface-coupled allergen, and the remaining group receives control vaccine comprising purified allergen. The vaccine is administered orally or nasally using an animal model as described in Repa et al. 2004 Clin Exp Immunol. 1:12-8. Alternatively, an allergy model is used where the mice is immunized initially with 5 μg/mL allergen combined with cholera toxin adjuvant to sensitize the animals to the allergen. Thereafter the mice are treated orally with the vaccine conjugates to de-sensitize the response. This is evaluated by spleenocyt activation assay as described in example 15 and evaluation of IgE antibodies. An allergy model for peanut allergy is described in Xiu-Min Li et al J allergy Clin Immunol 2000 106:1 and for B-lactoglobulin in Xiu-Min Li et al J allergy Clin Immunol 2001 107:4. These protocols are used in testing the present invention.

### Example 18

### Immunostimulatory Effect of the Untreated Bacteria and the Conjugates

Dendritic cells (DC) play a pivotal immunoregulatory role in the Th1, Th2, and Th3 cell balance and are present throughout the mucosal surfaces of an animal or human. Thus, DC may be targets for modulation by the vaccine conjugates. In the present example we have analysed the immune-stimulatory effect of the vaccine conjugates of the invention on DC in vitro. The DC model is used to select the bacterial strain that stimulates the desired immune response. Thus in the case of traditional pathogen vaccines, a bacterial strain with a high adjuvant effect is preferred. However, bacterial strains which polarize the immune system towards a Th1-type response may be desired for allergy vaccines, while bacterial strains favouring a strong CD8+ cytotoxic T cell response may be preferred in the development of a cancer vaccine. Similarly, bacterial strains inducing tolerance or anti-inflammation may be preferred in the design of vaccine conjugates for the treatment of auto-immune diseases.

### 18.1. Vaccine Conjugates

Vaccine conjugates containing surface-coupled beta-galactosidase to L. acidophilus X37 were prepared as described in example 10. Bone marrow cells were isolated and cultured as described by Lutz et al. J. Immunol. Methods 1999 223:77, with minor modifications. Briefly, femora and tibiae from two female C57BL/6 mice, 8-12 wk (Charles River Breeding Laboratories, Portage, Mich.), were removed and stripped of muscles and tendons. After soaking the bones in 70% ethanol for 2 min and rinsing in PBS, both ends were cut with scissors and the marrow was flushed with PBS using a 27-gauge needle. Cell clusters were dissociated by repeated pipetting. The resulting cell suspension was centrifuged for 10 min at 300×g and washed once in PBS. Cells were resuspended in RPMI 1640 (Sigma-Aldrich, St. Louis, Mo.) supplemented with 4 mM L-glutamine, 100 μg/ml penicillin, 100 μg/ml streptomycin, 50 μM 2-ME, 10% (v/v) heat-inactivated FBS (Atlanta Biologicals, Norcross, Ga.), and 15 ng/ml murine GM-CSF. GM-CSF was added as 5-10% (v/v) culture supernatant harvested from a GM-CSF-producing cell line (GM-CSF transfected Ag8.653 myeloma cell line) Zal et al. 1994 J. Immunol. Methods 223:77. The GM-CSF produced was quantified using a specific ELISA kit (BD PharMingen, San Diego, Calif.). To enrich for DC, 10 ml of cell suspension containing 3×10⁶ leukocytes was seeded per 100-mm bacteriological petri dish (day 0) and incubated for 8 days at 37° C. in an atmosphere of 5% CO₂. An additional 10 ml of freshly prepared medium was added to each plate on day 3. On day 6, 9 ml from each plate was centrifuged for 5 min at 300×g, and the resultant cell pellet was resuspended in 10 ml of fresh medium, and the suspension was returned to the dish. On day 8, cells were used to evaluate the effects of lactobacilli on cytokine release and expression of surface markers as described below.

### 18.2 Induction of Cytokine Release

Nonadherent cells were gently pipetted from petri dishes containing 8-day old DC-enriched cultures. The collected cells were centrifuged for 5 min at 300×g and resuspended in medium supplemented with only 10 ng/ml GM-CSF. Cells were seeded in 48-well tissue culture plates at 1.4×10⁶/500 μl/well, and then to each well was added (100 μl/well) one of the following solutions. a) conjugated L. acidophilus X37 (1-1000 μg/ml) solution with surface-coupled LacS, b) untreated L. acidophilus X37 (1-1000 μg/ml) solution, c) purified LacS B-galactosidase (prepared as described in example 3, at a similar LacS concentration as the LacS conjugate), d) LPS (Escherichia coli O26:B6; Sigma-Aldrich) at 1 μg/ml was added to some cultures as a positive control. Medium alone or medium containing 2-μm latex beads (Polysciences, Warrington, Pa.) were used as unstimulated and negative controls, respectively. After a stimulation period of 15 h at 37° C. in 5% CO₂, culture supernatant was collected and stored at −80° C. until cytokine analysis.

### 18.3 Cytokine Quantification in Culture Supernatants

IL-12(p70) and TNF-α were analyzed using commercially available ELISA kits (BD PharMingen) according to the manufacturer's instructions. IL-10 and IL-6 were similarly analyzed using matched Ab pairs purchased from BD PharMingen.

### 18.4 Results

Dendritic cells stimulated with vaccine conjugates showed IL-12 induction (FIG. 7). Furthermore, the induction of IL-12 increased with increasing conjugate concentration. The vaccine conjugates showed similar IL-12 induction to that of untreated L. acidophilus indicating that the adjuvant component of the bacteria is conserved in the vaccine conjugates. The protein (LacS) used for conjugation showed no immune induction alone.

1. A pharmaceutical composition for use as a medicament comprising a biological vehicle surface-displaying one or more heterologous proteinaceous compound including:
a. cells of one or more non-pathogenic bacterial strain, and b. one or more proteinaceous compound bound by means of a bi-functional cross-linker to an accessible chemical entity on the surface of said cells, wherein said cells do not comprise a transgenic nucleic acid molecule encoding said one or more proteinaceous compound, and said bi-functional linker is covalently bonded to an amino group of said cells via a Schiff-base, and said proteinaceous compound and said linker are heterologous in origin to said cells.

2. The composition of claim 1, wherein said bi-functional linker is selected from the group consisting of glutaraldehyde, polyazetidine and paraformaldehyde.

3. The composition of claim 1, wherein said biological vehicle comprises cells of either a non-genetically modified bacterial strain, or a genetically modified bacterial strain or a combination thereof.

4. The composition of claim 1, wherein said bacterial strain is a member of a bacterial genus selected from the group consisting Lactococcus, Lactobacillus, Leuconostoc, Group N Streptococcus, Enterococcus, Bifidobacterium, non-pathogenic Staphylococcus and non-pathogenic Bacillus.

5. The composition of claim 1, wherein said strain is a member of a bacterial genus selected from the group consisting of Lactobacillus and Bifidobacterium.

6. The composition of claim 1, wherein said strain is a member of a bacterial genus selected from Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovonis, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp delbrueckii, Lactobacillus delbrueckii subsp. lactis, Lactobacillus durianus, Lactobacillus equi, Lactobacillus farciminis, Lactobacillus ferintoshensis, Lactobacillus fermenturm, Lactobacillus formicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mali, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme, Lactobacillus zeae, Bifidobacterium adolescentis, Bifidobacterium aerophilum, Bifidobacterium angulatum, Bifidobacterium animalis, Bifidobacterium asteroides, Bifidobacterium bifidum, Bifidobacterium bourn, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum by Longum, Bifidobacterium longum by Infantis, Bifidobacterium longum by. Suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, and Bifidobacterium urinalis.

7. A composition according to claim 1, wherein said one or more proteinaceous compound is an antigen from an animal or human pathogen, or variant thereof.

8. A composition according to claim 7, wherein said animal or human pathogen is selected from the group consisting of from Poxyiridae, Herpesviridae, Adenoviridae, Parvoviridae, Papovaviridae, Hepadnaviridae, Picornaviridae, Caliciviridae, Reoviridae, Togaviridae, Flaviviridae, Arenaviridae, Retroviridae, Bunyaviridae, Orthomyxoviridae, Paramyxovihdae, Rhabdoviridae, Arboviruses, Oncoviruses, unclassified virus selected from Hepatitis viruses, Astrovirus and Torovirus, Bacillus, Mycobacterium, Plasmodium, Prions (e.g. causing Creutzfeldt-Jakob disease or variants), Cholera, Shigella, Escherichia, Salmonella, Corynebacterium, Borrelia, Haemophilus, Onchocerca, Bordetelia, Pneumococcus, Schistosoma, Clostridium, Chlamydia, Streptococcus, Staphylococcus, Campylobacter, Legionella, Toxoplasmose, Listeria, Vibrio, Nocardia, Clostridium, Neisseria, Candida, Trichomonas, Gardnerella, Treponema, Haemophilus, Klebsiella, Enterobacter, Proteus, Pseudomonas, Serratia, Leptospira, Epidermophyton, Microsporum, Trichophyton, Acremonium, Aspergillus, Candida, Fusarium, Scopulariopsis, Onychocola, Scytalidium, Histoplasma, Cryptococcus, Blastomyces, Coccidioides, Paracoccidioides, Zygomycetes, Sporothrix, Bordetella, Brucella, Pasteurella, Rickettsia, Bartonella, Yersinia, Giardia, Rhodococcus, Yersinia and Toxoplasma

9. A composition according to claim 1, wherein said one or more proteinaceous compound comprises an allergen of either extract, purified, recombinant, mutated or peptide source or variants thereof.

10. A composition according to claim 9, wherein the source of said allergen is selected from the group consisting of: birch trees, cats, cedar trees, olive trees, ragweed, other weeds, stinging insects, mosquitoes/midges, cockroaches, cattle, dogs, dust mites, grasses, outdoor moulds, indoor fungi, rodents, horses, nuts, milk, soy, wheat, eggs, shellfish and fish,

11. A composition according to claim 9, wherein the source of said allergen is selected from the group consisting of Birch pollen (Bet v), Grass pollen (Phl p, Lol p, Cyn d or Sor h), House Dust mite (Der p or Der f), Mite (Eur m, Blo t, Gly m, Lep d), Ragweed pollen (Amb a), Cedar Pollen (Cry j), Cat (Fel d), Wasp (Ves v or Dol m), Bee (Api m) and cockroach (Bla g, Per a).

12. A composition according to claim 9, wherein said allergen is a protein or peptide selected from the group consisting of Bet v 1, Bet v 2, Phl p 1, Phl p 5, Lol p 1 Lol p 5, Cyn d 1, Sor h 1, Der p 1 Der p2, Der f 1 or Der f 2, Eur m 1, Blo t 1, Gly m 1, Lep d 1, Amb a 1, Cry j 1, Fel d 1, Ves v 1, 2 or 5, Dol m 1, Dol m 2, Dol m 5, Api m 1, Bla g 1, and Per a 1.

13. A composition according to claim 1, wherein said one or more proteinaceous compound is an animal or human cancer antigen or variants thereof.

14. A composition according to claim 1, wherein said one or more proteinaceous compound is a self-antigen of animal or human origin, or variant thereof.

15. A composition according to claim 1, further comprising a spacer compound.

16. A composition according to claim 15, wherein said spacer is chitosan.

17. A composition according to any one of claim 1, wherein the number of molecules of proteinaceous compound bound per cell is in the range of 1 to about 100,000.

18. A composition according to any one of claim 1, wherein the number of molecules of proteinaceous compound bound per cell is in the range of 1 to about 10,000.

19. An encapsulated formulation comprising the composition according to claim 1.

20. Use of a composition according to claim 1, for the manufacture of a medicament for the prevention and/or treatment of a disease selected from the group consisting of: infectious disease, cancer, allergy, and autoimmune disease in an animal or human patient.

21. Use of a composition according to claim 1, for the manufacture of a medicament for the prevention and/or treatment of allergy in an animal or human patient.

22. Use of a composition according to claim 20, wherein said infectious disease is caused by an animal or human pathogen selected from the group consisting of from Poxyiridae, Herpesviridae, Adenoviridae, Parvoviridae, Papovaviridae, Hepadnaviridae, Picornaviridae, Caliciviridae, Reoviridae, Togaviridae, Flaviviridae, Arenaviridae, Retroviridae, Bunyaviridae, Orthomyxoviridae, Paramyxoviridae, Rhabdoviridae, Arboviruses, Oncoviruses, unclassified virus selected from Hepatitis viruses, Astrovirus and Torovirus, Bacillus, Mycobacterium, Plasmodium, Prions (e.g. causing Creutzfeldt-Jakob disease or variants), Cholera, Shigella, Escherichia, Salmonella, Corynebacterium, Borrelia, Haemophilus, Onchocerca, Bordetella, Pneumococcus, Schistosoma, Clostridium, Chlamydia, Streptococcus, Staphylococcus, Campylobacter, Legionella, Toxoplasmose, Listeria, Vibrio, Nocardia, Clostridium, Neisseria, Candida, Trichomonas, Gardnerella, Treponema, Haemophilus, Klebsiella, Enterobacter, Proteus, Pseudomonas, Serratia, Leptospira, Epidermophyton, Microsporum, Trichophyton, Acremonium, Aspergillus, Candida, Fusarium, Scopulariopsis, Onychocola, Scytalidium, Histoplasma, Cryptococcus, Blastomyces, Coccidioides, Paracoccidioides, Zygomycetes, Sporothrix, Bordetella, Brucella, Pasteurella, Rickettsia, Bartonella, Yersinia, Giardia, Rhodococcus, Yersinia and Toxoplasma

23. A method for prevention and/or treatment of a disease or allergy of an animal or human patient, wherein said patient is administered an effective dose of the composition according to claim 1.

24. A method according to claim 23, wherein said disease is selected from the group consisting of: infectious disease, cancer, allergy and autoimmune disease.

25. A method for the preparation of the pharmaceutical composition of claim 1, comprising a biological vehicle surface-displaying one or more heterologous proteinaceous compound, comprising the steps:
a. preparing a mixture comprising:
i. cells of one or more bacterial strain, and
ii. one or more heterologous proteinaceous compound, and
iii. a heterologous bi-functional cross-linker
b. incubating said mixture to form said biological vehicle in which said bi-functional linker is covalently bonded to an amino group of said cells via a Schiff-base, and
c. separating said biological vehicle from said mixture, wherein said cells do not comprise a transgenic nucleic acid molecule encoding said one or more proteinaceous compound.

26. The method according claim 25, wherein said bi-functional linker is selected from the group consisting of glutaraldehyde, polyazetidine and paraformaldehyde.

27. The method according to claim 25, wherein said mixture in step (b) is incubated at a temperature below 0° C.

28. The method according to claim 25, wherein said temperature is between −1° C. and −30° C.

29. A method according to claim 25, wherein said biological vehicle comprises cells of either a non-genetically modified bacterial strain, or a genetically modified bacterial strain.

30. A method according to claim 25, wherein said bacterial strain is a member of a bacterial genus selected from the group consisting Lactococcus, Lactobacillus, Leuconostoc, Group N Streptococcus, Enterococcus, Bifidobacterium, non-pathogenic Staphylococcus and non-pathogenic Bacillus.

31. The method according to claim 30, wherein said bacterial strain is a member of a bacterial genus selected from the group consisting of Lactobacillus and Bifidobacterium.

32. The method according to claim 31, wherein said bacterial strain is a member of bacterial species selected from Lactobacillus acetotolerans, Lactobacillus acidipiscis, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus arizonensis, Lactobacillus aviarius, Lactobacillus bifermentans, Lactobacillus brevis, Lactobacillus buchneri, Lactobacillus casei, Lactobacillus coelohominis, Lactobacillus collinoides, Lactobacillus coryniformis subsp. coryniformis, Lactobacillus coryniformis subsp. torquens, Lactobacillus crispatus, Lactobacillus curvatus, Lactobacillus cypricasei, Lactobacillus delbrueckii subsp. bulgaricus, Lactobacillus delbrueckii subsp. delbrueckii, Lactobacillus delbrueckii subsp. lactis, Lactobacillus durianus, Lactobacillus equi, Lactobacillus farciminis, Lactobacillus ferintoshensis, Lactobacillus fermentum, Lactobacillus formicalis, Lactobacillus fructivorans, Lactobacillus frumenti, Lactobacillus fuchuensis, Lactobacillus gallinarum, Lactobacillus gasseri, Lactobacillus graminis, Lactobacillus hamsteri, Lactobacillus helveticus, Lactobacillus helveticus subsp. jugurti, Lactobacillus heterohiochii, Lactobacillus hilgardii, Lactobacillus homohiochii, Lactobacillus intestinalis, Lactobacillus japonicus, Lactobacillus jensenii, Lactobacillus johnsonii, Lactobacillus kefiri, Lactobacillus kimchii, Lactobacillus kunkeei, Lactobacillus leichmannii, Lactobacillus letivazi, Lactobacillus lindneri, Lactobacillus malefermentans, Lactobacillus mall, Lactobacillus maltaromicus, Lactobacillus manihotivorans, Lactobacillus mindensis, Lactobacillus mucosae, Lactobacillus murinus, Lactobacillus nagelii, Lactobacillus oris, Lactobacillus panis, Lactobacillus pantheri, Lactobacillus parabuchneri, Lactobacillus paracasei subsp. paracasei, Lactobacillus paracasei subsp. pseudoplantarum, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus reuteri, Lactobacillus rhamnosus, Lactobacillus ruminis, Lactobacillus sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus thermophilus, Lactobacillus thermotolerans, Lactobacillus vaccinostercus, Lactobacillus vaginalis, Lactobacillus versmoldensis, Lactobacillus vitulinus, Lactobacillus vermiforme, Lactobacillus zeae, Bifidobacterium adolescentis, Bifidobacterium aerophilum, Bifidobacterium angulatum, Bifidobacterium animalis, Bifidobacterium asteroides, Bifidobacterium bifidum, Bifidobacterium bourn, Bifidobacterium breve, Bifidobacterium catenulatum, Bifidobacterium choerinum, Bifidobacterium coryneforme, Bifidobacterium cuniculi, Bifidobacterium dentium, Bifidobacterium gallicum, Bifidobacterium gallinarum, Bifidobacterium indicum, Bifidobacterium longum, Bifidobacterium longum by Longum, Bifidobacterium longum by. Infantis, Bifidobacterium longum by. Suis, Bifidobacterium magnum, Bifidobacterium merycicum, Bifidobacterium minimum, Bifidobacterium pseudocatenulatum, Bifidobacterium pseudolongum, Bifidobacterium pseudolongum subsp. globosum, Bifidobacterium pseudolongum subsp. pseudolongum, Bifidobacterium psychroaerophilum, Bifidobacterium pullorum, Bifidobacterium ruminantium, Bifidobacterium saeculare, Bifidobacterium scardovii, Bifidobacterium subtile, Bifidobacterium thermoacidophilum, Bifidobacterium thermoacidophilum subsp. suis, Bifidobacterium thermophilum, and Bifidobacterium urinalis.

33. A method according to claim 25, wherein said one or more proteinaceous compound is an antigen, from an animal or human pathogen, or variant thereof.

34. A method according to claim 33, wherein said animal or human pathogen is selected from the group consisting of Poxyiridae, Herpesviridae, Adenoviridae, Parvoviridae, Papovaviridae, Hepadnaviridae, Picornaviridae, Caliciviridae, Reoviridae, Togaviridae, Flaviviridae, Arenaviridae, Retroviridae, Bunyaviridae, Orthomyxoviridae, Paramyxoviridae, Rhabdoviridae, Arboviruses, Oncoviruses, unclassified virus selected from Hepatitis viruses, Astrovirus and Torovirus; Bacillus, Mycobacterium, Plasmodium, Prions (e.g. causing Creutzfeldt-Jakob disease or variants), Cholera, Shigella, Escherichia, Salmonella, Corynebacterium, Borrelia, Haemophilus, Onchocerca, Bordetella, Pneumococcus, Schistosoma, Clostridium, Chlamydia, Streptococcus, Staphylococcus, Campylobacter, Legionella, Toxoplasmose, Listeria, Vibrio, Nocardia, Clostridium, Neisseria, Candida, Trichomonas, Gardnerella, Treponema, Haemophilus, Klebsiella, Enterobacter, Proteus, Pseudomonas, Serratia, Leptospira, Epidermophyton, Microsporum, Trichophyton, Acremonium, Aspergillus, Candida, Fusarium, Scopulariopsis, Onychocola, Scytalidium, Histoplasma, Cryptococcus, Blastomyces, Coccidioides, Paracoccidioides, Zygomycetes, Sporothrix, Bordetella, Brucella, Pasteurella, Rickettsia, Bartonella, Yersinia, Giardia, Rhodococcus, Yersinia and Toxoplasma.

35. A method according to claim 25, wherein said one or more proteinaceous compound is an allergen of either extract, purified, recombinant, mutated or peptide source or variants thereof.

36. A method according to claim 33, wherein the source of said allergen is selected from the group consisting of: birch trees, cats, cedar trees, olive trees, ragweed, other weeds, stinging insects, mosquitoes/midges, cockroaches, cattle, dogs, dust mites, grasses, outdoor moulds, indoor fungi, rodents, horses, nuts, milk, soy, wheat, eggs, shellfish, and fish.

37. A method according to claim 35, wherein the source of said allergen is selected from the group consisting of Birch pollen (Bet v), Grass pollen (Phi p, Lol p, Cyn d or Sor h), House Dust mite (Der p or Der f), Mite (Eur m, Blo t, Gly m, Lep d), Ragweed pollen (Amb a), Cedar Pollen (Cry j), Cat (Fel d), Wasp (Ves v or Dol m), Bee (Api m) and cockroach (Bla g, Per a).

38. A method according to claim 35, wherein said allergen is a protein or peptide selected from the group consisting of Bet v 1, Bet v 2, Phl p 1, Phl p 5, Lol p 1 Lol p 5, Cyn d 1, Sor h 1, Der p 1 Der p 2, Der f 1 or Der f 2, Eur m 1, Blo t 1, Gly m 1, Lep d 1, Amb a 1, Cry j 1, Fel d 1, Ves v 1, 2 or 5, Dol m 1, Dol m 2, Dol m 5, Api m 1, Bla g 1, and Per a 1.

39. A method according to claim 25, wherein said one or more proteinaceous compound is an animal or human cancer antigen or variant thereof.

40. A method according to claim 25, wherein said one or more proteinaceous compound is a self-antigen of animal or human origin, or variant thereof.

41. A method according to claim 25, wherein said mixture further comprising a spacer compound.

42. A method according to claim 37, wherein said spacer is chitosan.

43. A method according to claim 23, wherein the number of molecules of proteinaceous compound bound per cell is between 1 and about 100,000.

44. A method according to claim 23, wherein the number of molecules of proteinaceous compound bound per cell is between 1- and about 10,000.

45. A method according to claim 23, further comprising the step of encapsulating said biological vehicle.
